(19) United States
(12) Patent Application Publication
(10) Pub. No.: US 2023/0322834 A1
(43) Pub. Date: Oct. 12, 2023

(54) POLYMERIZATION AND FUNCTIONALIZATION OF LEVOGLUCOSAN DERIVATIVES

(71) Applicant: Regents of the University of Minnesota, Minneapolis, MN (US)

(72) Inventors: Theresa M. Reineke, Minneapolis, MN (US); Christopher J. Ellison, Eden Prairie, MN (US); Mayuri Kiran Porwal, Minneapolis, MN (US); Derek J. Saxon, Minneapolis, MN (US)

(21) Appl. No.: 18/131,673

(22) Filed: Apr. 6, 2023

Related U.S. Application Data
(60) Provisional application No. 63/328,114, filed on Apr. 6, 2022.

(51) Int. Cl. C07H 1/08; C07H 3/10

(52) U.S. Cl. CPC C07H 1/08; C07H 3/10

(57) ABSTRACT

Provided herein are polymers derived from levoglucosan, methods of preparing said polymers, and methods of modifying said polymers.

### CROSS-REFERENCE TO RELATED APPLICATIONS

This application claims the benefit of U.S. Application Ser. No. 63/328,114 entitled “POLYMERIZATION AND FUNCTIONALIZATION OF LEVOGLUCOSAN DERIVATIVES” filed Apr. 6, 2022, which is incorporated by reference herein in its entirety.

### STATEMENT OF GOVERNMENT INTEREST

This invention was made with government support under CHE-1901635 awarded by the National Science Foundation. The government has certain rights in the invention.

### TECHNICAL FIELD

This invention relates to compounds derived from biomass and methods for preparing and functionalizing said compounds.

### BACKGROUND

Lignocellulosic biomass is one of the most promising renewable feedstocks for sustainable polymers due to its worldwide abundance and availability. The major component of lignocellulosic biomass is cellulose, and thermochemical conversion processes-such as fast pyrolysis-provide an efficient route to convert biomass cellulose into renewable chemicals. The major product of cellulose fast pyrolysis is levoglucosan. Structurally, levoglucosan is an attractive feedstock for the synthesis of sustainable polymers. Therefore, there is a need for the preparation of sustainable polymers derived from levoglucosan.

### SUMMARY

Provided herein are compounds derived from biomass and methods for preparing and functionalizing said compounds.

Embodiment 1 is a compound according to Formula (I):

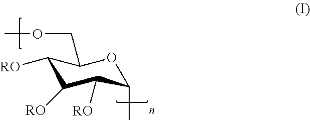

wherein:
each R is independently C₂-C₂₀ alkyl, C₂-C₂₀ alkenyl, C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl comprise 1-5 heteroatoms selected from N, O, and S; and
n is in a range of 3 to 1000;
with the proviso that each R cannot be benzyl.

Embodiment 2 is the compound of embodiment 1, wherein at least one R is independently C₂-C₂₀ alkenyl, C₂-C₂₀ alkynyl, C₁-C₂₀ heteroalkyl, C₁-C₈ heterocycloalkyl, or C₁-C₁₅ heteroaryl.

Embodiment 3 is the compound of embodiment 1 or 2, wherein each R is independently C₂-C₂₀ alkenyl, C₂-C₂₀ alkynyl, C₁-C₂₀ heteroalkyl, C₁-C₈ heterocycloalkyl, or C₁-C₁₅ heteroaryl.

Embodiment 4 is the compound of any one of embodiments 1-3, wherein at least one R is C₂-C₂₀ alkenyl or C₁-C₂₀ heteroalkyl.

Embodiment 5 is the compound of any one of embodiments 1-4, wherein each R is independently C₂-C₂₀ alkenyl or C₁-C₂₀ heteroalkyl.

Embodiment 6 is the compound of any one of embodiments 1-5, wherein at least one R is C₂-C₂₀ alkenyl.

Embodiment 7 is the compound of any one of embodiments 1-6, wherein each R is C₂-C₂₀ alkenyl.

Embodiment 8 is the compound of any one of claims 1-5, wherein at least one R is C₁-C₂₀ heteroalkyl.

Embodiment 9 is the compound of any one of embodiments 1-5 or 8, wherein each R is C₁-C₂₀ heteroalkyl.

Embodiment 10 is the compound of any one of embodiments 1-5, wherein at least one R is

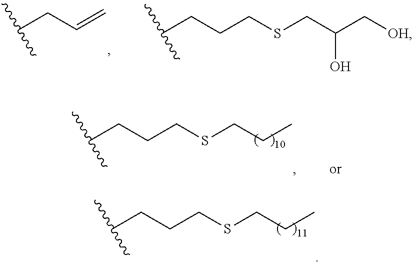

Embodiment 11 is the compound of any one of embodiments 1-5 or 10, wherein each R is

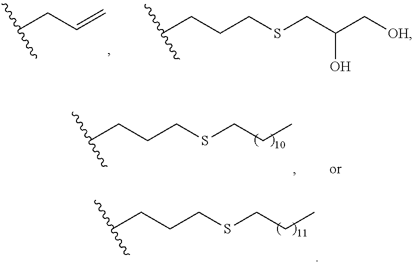

Embodiment 12 is the compound of any one of embodiments 1-5 or 10, wherein at least one R is

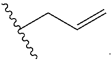 [SMILES: *CC=C]

Embodiment 13 is the compound of any one of embodiments 1-5, 10, or 12, wherein each R is

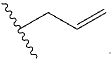 [SMILES: *CC=C]

Embodiment 14 is a method of ring-opening polymerization, the method comprising: combining a catalyst and a compound according to Formula (Ia):

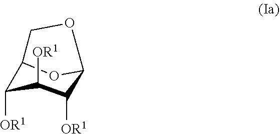

to form a compound according to Formula (I):

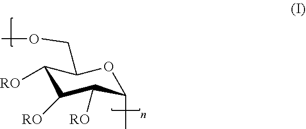

wherein:
each R is independently C₂-C₂₀ alkyl, C₂-C₂₀ alkenyl, C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl comprise 1-5 heteroatoms selected from N, O, and S;
each R¹ is independently C₂-C₂₀ alkyl, C₂-C₂₀ alkenyl, C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl comprise 1-5 heteroatoms selected from N, O, and S; and
n is in a range of 3 to 1000;
with the proviso that each R and each R¹ cannot be benzyl.

Embodiment 15 is the method of embodiment 14, wherein at least one R and at least one R¹ is independently C₂-C₂₀ alkenyl, C₂-C₂₀ alkynyl, C₁-C₂₀ heteroalkyl, C₁-C₈ heterocycloalkyl, or C₁-C₁₅ heteroaryl.

Embodiment 16 is the method of embodiment 14 or 15, wherein each R and each R¹ is independently C₂-C₂₀ alkenyl, C₂-C₂₀ alkynyl, C₁-C₂₀ heteroalkyl, C₁-C₈ heterocycloalkyl, or C₁-C₁₅ heteroaryl.

Embodiment 17 is the method of any one of embodiments 14-16, wherein at least one R and at least one R¹ is C₂-C₂₀ alkenyl or C₁-C₂₀ heteroalkyl.

Embodiment 18 is the method of any one of embodiments 14-17, wherein each R and each R¹ is independently C₂-C₂₀ alkenyl or C₁-C₂₀ heteroalkyl.

Embodiment 19 is the method of any one of embodiments 14-18, wherein at least one R and at least one R¹ is C₂-C₂₀ alkenyl.

Embodiment 20 is the method of any one of embodiments 14-19, wherein each R and each R¹ is C₂-C₂₀ alkenyl.

Embodiment 21 is the method of any one of embodiments 14-18, wherein at least one R and at least one R¹ is C₁-C₂₀ heteroalkyl.

Embodiment 22 is the method of any one of embodiments 14-18 or 21, wherein each R and each R¹ is C₁-C₂₀ heteroalkyl.

Embodiment 23 is the method of any one of embodiments 14-18, wherein at least one R and at least one R¹ is

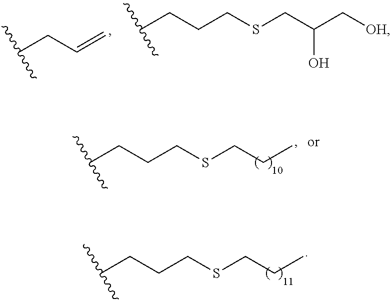

Embodiment 24 is the method of any one of embodiments 14-18, wherein each R and each R¹ is

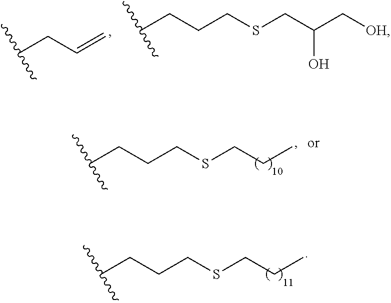

Embodiment 25 is the method of any one of embodiments 14-18 or 23, wherein at least one R and at least one R¹ is

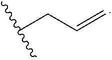 [SMILES: *CC=C]

Embodiment 26 is the method of any one of embodiments 14-18, wherein each R and each R¹ is

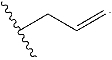 [SMILES: *CC=C]

Embodiment 27 is the method of any one of embodiments 14-26, wherein the catalyst is a Lewis acid.

Embodiment 28 is the method of embodiment 27, wherein the Lewis acid is a triflate.

Embodiment 29 is the method of embodiments 27 or 28, wherein the Lewis acid is MeOTf, Sc(OTf)₃, or Bi(OTf)₃.

Embodiment 30 is the method of any one of embodiments 14-29, wherein the catalyst is present in an amount in a range of about 0.01 mol % to about 5 mol %, based on the molar amount of the compound according to Formula (Ia).

Embodiment 31 is the method of any one of embodiments 14-30 further comprising combining levoglucosan, a base, and a reactant to form the compound according to Formula (Ia).

Embodiment 32 is a method of modifying a polymer, the method comprising:
combining a thiol or an azide, and a compound according to Formula (I):

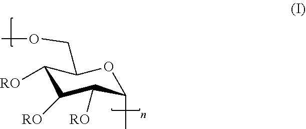

to form a compound according to Formula (II):

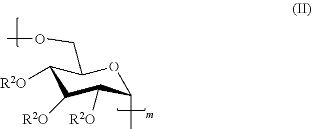

wherein:
each R is independently C₂-C₂₀ alkenyl, C₂-C₂₀ alkynyl, C₁-C₂₀ heteroalkyl, C₁-C₈ heterocycloalkyl, C₁-C₁₅ heteroaryl and each of the heteroalkyl, heterocycloalkyl, and heteroaryl comprise 1-5 heteroatoms selected from N, O, and S;
each R² is independently C₂-C₂₀ alkyl, C₂-C₂₀ alkylene, C₂-C₂₀ alkylyne, C₁-C₂₀ heteroalkyl, C₁-C₈ heterocycloalkyl, C₂-C₂₀ heteroalkylene, C₂-C₂₀ heteroalkylyne, C₃-C₈ cycloalkyl, C₆-C₂₀ aryl, C₁-C₁₅ heteroaryl and each of the heteroalkyl, heterocycloalkyl, heteroalkylene, heteroalkylyne, and heteroaryl comprise 1-5 heteroatoms selected from N, O, and S;
at least one R² is a C₁-C₂₀ thioalkyl or C₁-C₁₅ heteroaryl; and
n and m are each independently in a range of 3 to 1000.

Embodiment 33 is the method of embodiment 32, wherein at least one R is C₂-C₂₀ alkenyl or C₂-C₂₀ alkynyl.

Embodiment 34 is the method of embodiment 32 or 33, wherein each R is C₂-C₂₀ alkenyl or C₂-C₂₀ alkynyl.

Embodiment 35 is the method of any one of claims 32-34, wherein at least one R is

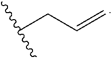 [SMILES: *CC=C]

Embodiment 36 is the method of any one of embodiments 32-35, wherein each R is

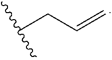 [SMILES: *CC=C]

Embodiment 37 is the method of any one of embodiments 32-36, wherein each R² is a C₁-C₂₀ thioalkyl.

Embodiment 38 is the method of any one of embodiments 32-37, wherein at least one R² is

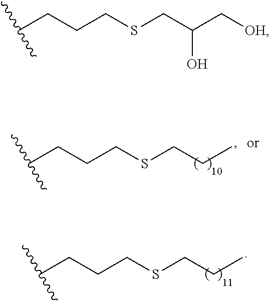

Embodiment 39 is the method of any one of embodiments 32-38, wherein each R² is

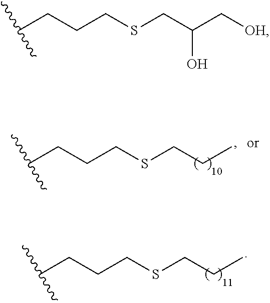

Embodiment 40 is the method of any one of embodiments 32-34, wherein at least one R² is C₁-C₁₅ heteroaryl.

Embodiment 41 is the method of embodiment 40, wherein each R² is C₁-C₁₅ heteroaryl.

Embodiment 42 is the method of embodiment 40 or 41, wherein the heteroaryl is a triazolyl.

### BRIEF DESCRIPTION OF FIGURES

FIG. 1 depicts a graphic of converting biomass to levogluosan then cationic ring-opening polymerization to tailorable stereoregular polysaccharides.

FIG. 2a depicts an example of a synthesis of levoglucosan from cellulosic biomass through a functionalized polysaccharide.

FIG. 2b depicts an isodesmic reaction for ring-opening of 2 and 3 with dimethyl ether and the corresponding ring strain free energies (kcal/mol) at the SMD(DCM)/M06-2X/6-311++G(d,p)/SMD(DCM)/M06-L/6-31+G(d,p) level.

FIG. 3 depicts a summary of the cationic ring-opening polymerizations (cROP) of 2 and 3 under various reaction conditions.^{a}Monomer conversion determined by ¹H NMR spectroscopy. ^{b}Molecular weight and dispersity determined by SEC-MALS in dimethylformamide.

FIG. 4a depicts key mechanistic steps for cROP of 2 and 3 catalyzed by Lewis acid. Transition-state structures for the ring-opening of 1,6-anhydro linkages (TS1) and nucleophilic additions of monomers 2 and 3 (TS2) catalyzed by various Lewis acids. Computed energetics indicate that nucleophilic addition of monomers 2 or 3 to 12 to form intermediate 14 (pseudo-equatorial approach) is energetically disfavored compared to formation of intermediate 13.

FIG. 4b is a table depicting computed energetics in kcal/mol for key mechanistic steps.

FIG. 4c is an alternative pathway for cROP of 2 catalyzed by Sc(OTf)₃ and the cROP of 3 catalyzed by Bi(OTf)₃. The corresponding free energies of activation (kcal/mol) with respect to I1 at the SMD(DCM)/ωB97X-D/def2-TZVP, def2-TZVP|SDD(Bi)/SMD(DCM)/M06-L/6-31+G(d,p), LanL2DZ(Bi) level.

FIG. 5a is a graph of the conversion-time plot for cROP of 2 with Sc(OTf)₃. Polymerization conditions: [2]0=2M, 2: Sc(OTf)₃=200:1. Monomer conversion determined by ¹H NMR spectroscopy. Error bars based on true values.

FIG. 5b is a graph of the conversion-time plot for cROP of 3 with Bi(OTf)₃. Polymerization conditions: [3]0=7M, 3: Bi(OTf)₃=100:1. Monomer conversion determined by ¹H NMR spectroscopy. Error bars based on true values.

FIG. 5c is a depiction of the coordination modes for monomer 3 with a Lewis acid (LA), Bi(OTf)₃, and the corresponding binding free energies (AGI in kcal/mol) are provided in the parenthesis.

FIG. 6a is a schematic depicting post-polymerization modification of poly(3) by UV-initiated thiol-ene reaction with 1-thioglycerol and lauryl mercaptan to create poly(4) and poly(5) respectively.

FIG. 6b is a graph of the post-polymerization modification kinetic data depicting conversion of the C═C functional groups in poly(3) during UV irradiation. The ratio of thiol to ene groups in both the formulations is 1:1.

FIG. 6c is a graph of the thermogravimetric analysis (TGA) curves (under N₂, 10° C./min) of the levoglucosan-based polymers and control dextran.

FIG. 6d is a graph of the differential scanning calorimetry (DSC) thermogram (second heating, 10° C./min) of poly(5) depicting the double melting peak.

FIG. 7 is a graph of the conversion versus time plot of two replicates of the cROP of 2.

FIG. 8 is a graph of the conversion versus time plot of three replicates of the cROP of 3.

FIG. 9 is a graph of the molecular weight (Mₙ, g/mol) versus conversion for the cROP of 2.

FIG. 10 is a graph of the molecular weight (Mₙ, g/mol) versus conversion for the cROP of 3.

FIG. 11 is a graph of the dispersity (D) versus conversion for the cROP of 2.

FIG. 12 is a graph of the dispersity (D) versus conversion for the cROP of 3.

FIG. 13 is a Fourier transform infrared (FTIR) spectra of poly(3)-thioglycerol as a function of irradiation time.

FIG. 14 is a FTIR spectra of poly(3)-lauryl mercaptan as a function of irradiation time.

FIG. 15 is a DSC thermogram (second heating, 10° C./min) for poly(2).

FIG. 16 is a DSC thermogram (second heating, 10° C./min) for poly(3).

FIG. 17 is a DSC thermogram (second heating, 10° C./min) for poly(4).

FIG. 18 is a DSC thermogram (second heating, 20° C./min) for poly(5).

FIG. 19 is a size-exclusion chromatography (SEC) trace of poly(2).

FIG. 20 is a SEC trace of poly(3).

FIG. 21 is a SEC trace of poly(4).

FIG. 22 is a SEC trace of poly(5).

FIGS. 23A and 23B are graphs of the green fluorescent protein (GFP) knockdown percentage and the cell viability percentage of cells when delivered antisense oligonucleotides (ASOs), lipofectamine (L2K), and a polymer as disclosed herein with ASOs.

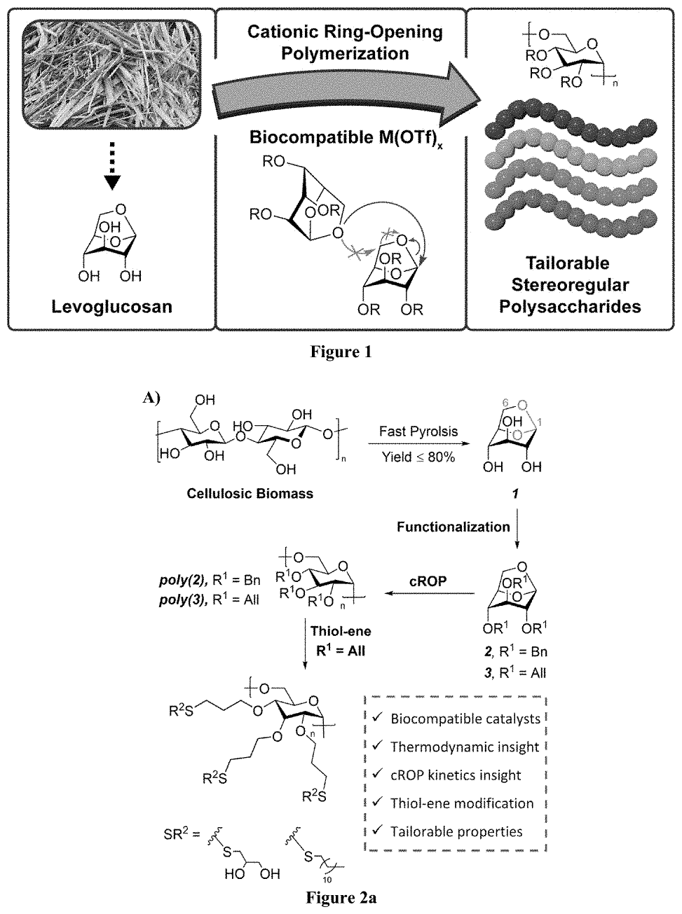

### DETAILED DESCRIPTION

Provided herein are polymers according to the disclosure, methods of ring-opening polymerization, and methods of modifying a polymer.

Advantageously, disclosed herein is a synthetic platform for stereoregular 1,6-α linked levoglucosan-based polysaccharides with different pendant functional groups. Levoglucosan derivatives have been polymerized using biocompatible and recyclable metal triflate catalysts—scandium and bismuth triflate—for green cROP of levoglucosan derivatives, even at very low catalyst loadings of 0.5 mol %. Post-polymerization modification of levoglucosan-based polysaccharides was readily performed via UV-initiated thiol-ene click reactions. The disclosed levoglucosan based polymers exhibit good thermal stability (T_{d}>250° C.) and a wide glass transition temperature (T_{g}) window (<−150° C. to 32° C.) that is accessible with thioglycerol and lauryl mercaptan pendant groups. The disclosure herein demonstrated the utility of levoglucosan as a renewably-derived scaffold, enabling facile access to tailored polysaccharides that could be important in many applications ranging from sustainable materials to biologically active polymers.

Compounds Derived from Levoglucosan

The present disclosure provides polymers derived from levoglucosan. In some embodiments, the polymer can include compounds according to Formula (I):

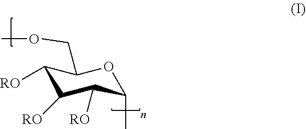

wherein:
each R is independently C₂-C₂₀ alkyl, C₂-C₂₀ alkylene, C₂-C₂₀ alkylyne, C₁-C₂₀ heteroalkyl, C₁-C₈ heterocycloalkyl, C₂-C₂₀ heteroalkylene, C₂-C₂₀ heteroalkylyne, C₃-C₈ cycloalkyl, C₆-C₂₀ aryl, C₁-C₁₅ heteroaryl and each of the heteroalkyl, heterocycloalkyl, heteroalkylene, heteroalkylyne, and heteroaryl include 1-5 heteroatoms selected from N, O, and S; and
n is in a range of 3 to 1000;
with the proviso that each R cannot be benzyl.

As used herein, the term “alkyl” refers to straight chained and branched saturated hydrocarbon groups containing one to thirty carbon atoms, for example, one to twenty carbon atoms, or one to ten carbon atoms. The term C₁ means the alkyl group has “n” carbon atoms. For example, C₄ alkyl refers to an alkyl group that has 4 carbon atoms. C₂-C₂₀ alkyl refers to an alkyl group having a number of carbon atoms encompassing the entire range (i.e., 2 to 20 carbon atoms), as well as all subgroups (e.g., 2-19, 2-15, 2-10, 2-8, 2, 3, 4, 5, 6, 7, 10, 12, 14, 16, 18, carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

As used herein, the term “alkenyl” refers to a saturated straight chain or branched non-cyclic hydrocarbon having from 2 to 20 carbon atoms and having at least one carbon-carbon double bond. Representative straight chain and branched C₂-C₂₀ alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Unless otherwise indicated, a alkenyl group can be an unsubstituted alkenyl group or a substituted alkenyl group.

As used herein, the term “alkynyl” refers to a saturated straight chain or branched non-cyclic hydrocarbon having from 2 to 20 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Unless otherwise indicated, an alkynyl group can be an unsubstituted alkynyl group or a substituted alkynyl group.

As used herein, the term “heteroalkyl” is defined similarly as alkyl, except the alkyl chain contains one to three heteroatoms independently selected from oxygen, nitrogen, or sulfur and the heteroalkyl can be saturated or unsaturated. For example, the heteroalkyl can include an alkene bond or an alkyne bond. For example, the heteroalkyl can be a thioalkyl, amine, ester, carbonate, carbamate, or the like. Unless otherwise indicated, a heteroalkyl group can be an unsubstituted heteroalkyl group or a substituted heteroalkyl group.

As used herein, the term “cycloalkyl” refers to an aliphatic cyclic hydrocarbon group containing three to eight carbon atoms (e.g., 3, 4, 5, 6, 7, or 8 carbon atoms). The term C₁ means the cycloalkyl group has “n” carbon atoms. For example, C₅ cycloalkyl refers to a cycloalkyl group that has 5 carbon atoms in the ring. C₅-C₈ cycloalkyl refers to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group.

As used herein, the term “heterocycloalkyl” is defined similarly as cycloalkyl, except the ring contains one to three heteroatoms independently selected from oxygen, nitrogen, or sulfur. In particular, the term “heterocycloalkyl” refers to a ring containing a total of three to eight atoms, of which 1, 2, 3 or three of those atoms are heteroatoms independently selected from the group consisting of oxygen, nitrogen, and sulfur, and the remaining atoms in the ring are carbon atoms. Nonlimiting examples of heterocycloalkyl groups include piperdine, tetrahydrofuran, tetrahydropyran, dihydrofuran, morpholine, and the like. Heterocycloalkyl groups can be saturated or partially unsaturated ring systems optionally substituted with, for example, one to three groups, independently selected alkyl, alkylene, OH, C(O)NH₂, NH₂, oxo (═O), aryl, haloalkyl, halo, and OH. Heterocycloalkyl groups optionally can be further N-substituted with alkyl, hydroxyalkyl, alkylene-aryl, and alkylene-heteroaryl. The heterocycloalkyl groups described herein can be isolated or fused to another heterocycloalkyl group, a cycloalkyl group, an aryl group, and/or a heteroaryl group. When a heterocycloalkyl group is fused to another heterocycloalkyl group, then each of the heterocycloalkyl groups can contain three to eight total ring atoms, and one to three heteroatoms. In some embodiments, the heterocycloalkyl groups described herein include one oxygen ring atom (e.g., oxiranyl, oxetanyl, tetrahydrofuranyl, and tetrahydropyranyl).

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “heteroaryl” refers to a cyclic aromatic ring having five to twelve total ring atoms (e.g., a monocyclic aromatic ring with 5-6 total ring atoms), and containing one to three heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Heteroaryl groups can be isolated (e.g., pyridyl) or fused to another heteroaryl group (e.g., purinyl), a cycloalkyl group (e.g., tetrahydroquinolinyl), a heterocycloalkyl group (e.g., dihydronaphthyridinyl), and/or an aryl group (e.g., benzothiazolyl and quinolyl). Examples of heteroaryl groups include, but are not limited to, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, alkynyl, heteroalkyl, heterocycloalkyl, ether, polyether, thioalkyl, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

The phrase “therapeutically effective amount” refers an amount of compound that, when administered to a subject in need of such treatment, is sufficient to (i) treat a particular disease, (ii) attenuate, ameliorate, or eliminate one or more symptoms of the particular disease, or (iii) delay the onset of one or more symptoms of the particular disease described herein.

The term “pharmaceutically acceptable salt” refers to a formulation of a compound that does not cause significant irritation to an organism to which it is administered and does not abrogate the biological activity and properties of the compound.

As used herein, the term “subject” refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease to be treated and/or prevented.

In some embodiments, at least one R is independently C₂-C₂₀ alkenyl, C₂-C₂₀ alkynyl, C₁-C₂₀ heteroalkyl, C₁-C₈ heterocycloalkyl, or C₁-C₁₅ heteroaryl. In some embodiments, each R is independently C₂-C₂₀ alkenyl, C₂-C₂₀ alkynyl, C₁-C₂₀ heteroalkyl, C₁-C₈ heterocycloalkyl, or C₁-C₁₅ heteroaryl.

In some embodiments, at least one R is C₂-C₂₀ alkenyl or C₁-C₂₀ heteroalkyl. In some embodiments, each R is independently C₂-C₂₀ alkenyl or C₁-C₂₀ heteroalkyl

In some embodiments, at least one R is C₂-C₂₀ alkenyl. In some embodiments, each R is C₂-C₂₀ alkenyl.

In some embodiments, at least one R is C₁-C₂₀ heteroalkyl. In some embodiments, each R is C₁-C₂₀ heteroalkyl. In some embodiments, R is C₁-C₂₀ heteroalkyl and wherein each C₁-C₂₀ heteroalkyl comprises 1-3 heteroatoms selected from S, N, and O. In some embodiments, R is C₁-C₂₀ heteroalkyl and wherein each C₁-C₂₀ heteroalkyl comprises 1-3 heteroatoms selected from S and N. In some embodiments, R is C₆-C₁₆ heteroalkyl and wherein each C₆-C₁₆ heteroalkyl comprises 1-3 heteroatoms selected from S and N. In some embodiments, R is C₆-C₁₆ heteroalkyl and wherein each C₆-C₁₆ heteroalkyl comprises 1 heteroatom and the heteroatom is S. In some embodiments, R is C₆-C₁₆ heteroalkyl and wherein each C₆-C₁₆ heteroalkyl comprises 2 heteroatoms selected from S and N. In some embodiments, R is C₆-C₁₆ heteroalkyl and wherein each C₆-C₁₆ heteroalkyl comprises 2 heteroatoms selected from S and O. In some embodiments, R is C₆-C₁₆ heteroalkyl and wherein each C₆-C₁₆ heteroalkyl comprises 3 heteroatoms selected from S and O. In some embodiments, R is C₆-C₁₆ heteroalkyl and wherein each C₆-C₁₆ heteroalkyl comprises 3 heteroatoms selected from S and N. In some embodiments, R is C₁-C₂₀ heteroalkyl and wherein each C₁-C₂₀ heteroalkyl comprises 1-3 heteroatoms and the heteroatoms are N. In some embodiments, R is C₁-C₁₀ heteroalkyl and wherein each C₁-C₁₀ heteroalkyl comprises 1-2 heteroatoms and the heteroatoms are N. In some embodiments, R is C₁-C₂₀ heteroalkyl and wherein each C₁-C₂₀ heteroalkyl comprises 1-3 heteroatoms and the heteroatoms are O. In some embodiments, R is C₁-C₁₀ heteroalkyl and wherein each C₁-C₁₀ heteroalkyl comprises 1-2 heteroatoms and the heteroatoms are O.

In some embodiments, at least one R is

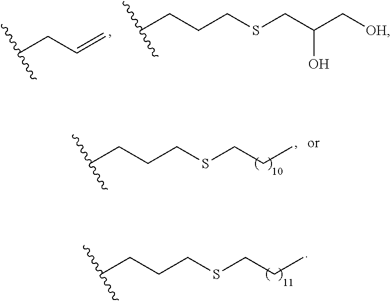

In some embodiments, each R is

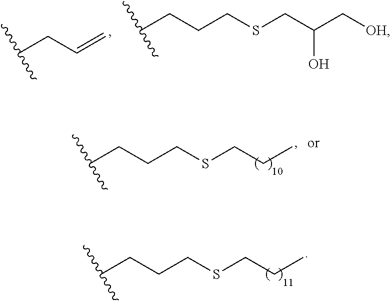

In some embodiments, at least one R is

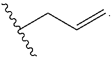 [SMILES: *CC=C]

In some embodiments, each R is

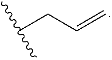 [SMILES: *CC=C]

In some embodiments, each R is independently C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl comprise 1-5 heteroatoms selected from N, O, and S.

In some embodiments, at least one R is independently C₂-C₂₀ alkynyl, C₁-C₂₀ heteroalkyl, C₁-C₈ heterocycloalkyl, or C₁-C₁₅ heteroaryl. In some embodiments, each R is independently C₂-C₂₀ alkynyl, C₁-C₂₀ heteroalkyl, C₁-C₈ heterocycloalkyl, or C₁-C₁₅ heteroaryl.

In some embodiments, at least one R is

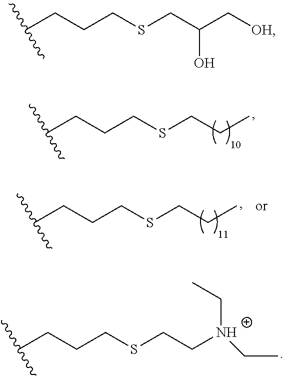

In some embodiments, each R is

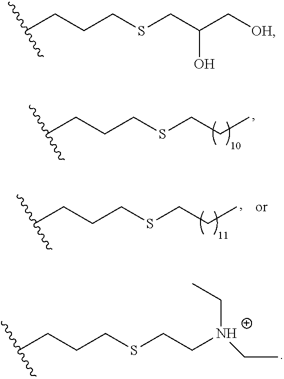

In some embodiments, R is

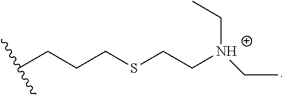 [SMILES: *CCCSCC[NH+](CC)CC]

In some embodiments, at least one R is cationic. In some embodiments, each R is cationic. In some embodiments, at least one R comprises a cationic amine. For example, the cationic amine can include, but is not limited to, —(N(CH₃)₃)⁺, —(NH₃)⁺, —(NH(CH₂CH₃)₂)⁺, —(NH(CH₃)₂)⁺, and

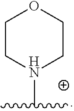

In some embodiments, a compound according to Formula (I) as disclosed herein is cationic.

In some embodiments, n can in a range of 3 to 1000. For example, n can in a range of about 5 to about 500, or about 5 to about 400, or about 10 to about 250, or about 20 to about 150, or about 25 to about 100, or about 20 to about 75, or about 30 to 60.

The compounds according to Formula (I) can be characterized by the number average molecular weight (Mₙ) or the weight average molecular weight (M_{w}). In some embodiments, the compounds according to Formula (I) can have a Mₙ in a range of about 1000 to about 500,000. For example, the compounds according to Formula (I) can have a Mₙ in a range of about 1000 to about 250,000, or about 1000 to about 150,000, or about 5000 to about 100,000, or about 2000 to about 20,000. In some embodiments, the compounds according to Formula (I) can have a Mₙ in a range of about 1000 to about 100,000.

Methods of Preparing Compounds according to Formula (I)

The present disclosure provides methods of ring-opening polymerizations of levoglucosan derived monomers. In some embodiments the method includes combining a catalyst and a compound according to Formula (Ia):

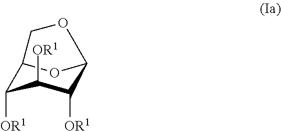

to form a compound according to Formula (I) disclosed above, wherein each R¹ is independently C₂-C₂₀ alkyl, C₂-C₂₀ alkenyl, C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl includes 1-5 heteroatoms selected from N, O, and S, with the proviso that each R and R¹ cannot be benzyl.

In some embodiments, at least one R and at least one R¹ is independently C₂-C₂₀ alkenyl, C₂-C₂₀ alkynyl, C₁-C₂₀ heteroalkyl, C₁-C₈ heterocycloalkyl, or C₁-C₁₅ heteroaryl. In some embodiments, each R and each R¹ is independently C₂-C₂₀ alkenyl, C₂-C₂₀ alkynyl, C₁-C₂₀ heteroalkyl, C₁-C₁₅ heterocycloalkyl, or C₁-C₁₅ heteroaryl.

In some embodiments, at least one R and at least one R¹ is C₂-C₂₀ alkenyl or C₁-C₂₀ heteroalkyl. In some embodiments, each R and each R¹ is independently C₂-C₂₀ alkenyl or C₁-C₂₀ heteroalkyl.

In some embodiments, at least one R and at least one R¹ is C₂-C₂₀ alkenyl. In some embodiments, each R and each R¹ is C₂-C₂₀ alkenyl.

In some embodiments, at least one R and at least one R¹ is C₁-C₂₀ heteroalkyl. In some embodiments, each R and each R¹ is C₁-C₂₀ heteroalkyl.

In some embodiments at least one R and at least one R¹ is

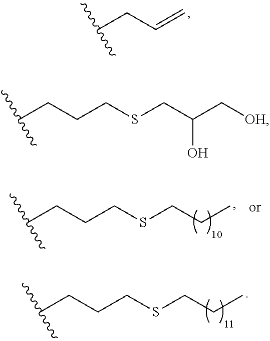

In some embodiments, each R and each R¹ is

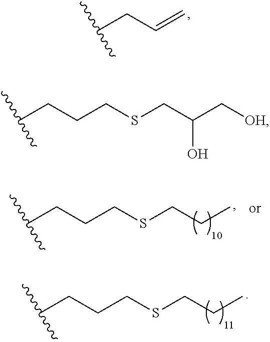

In some embodiments, at least one R and at least one R¹ is

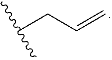 [SMILES: *CC=C]

In some embodiments, each R and each R¹ is

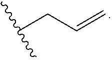 [SMILES: *CC=C]

In some embodiments, the catalyst can be a Lewis acid. In some embodiments, the Lewis acid is a triflate. In some embodiments, the Lewis acid is one or more of bismuth subsalicylate, diphenyl phosphoric acid, Ti(iOPr)₄, Al(iOPr)₃, BF₃OEt₂, MeOTf, Fe(OTf)₃, Sc(OTf)₃, Al(OTf)₃, Bi(OTf)₃, La(OTf)₃, Y(OTf)₃, and Zn(OTf)₂. In some embodiments, the Lewis acid is MeOTf, Sc(OTf)₃, or Bi(OTf)₃.

The methods disclosed herein can include a catalyst loading of up to 20 mol %, based on the molar amount of the compound according to Formula (Ia). In some embodiments, the catalyst loading can be in a range of about 0.01 mol % to about 5 mol %, based on the molar amount of the compound according to Formula (Ia). In some embodiments, the catalyst loading can be up to 10 mol %, 9 mol %, 8 mol %, 7 mol %, 6 mol %, 5 mol %, 4 mol %, 3 mol %, 2 mol %, 1 mol %, 0.5 mol %, 0.1 mol %, 0.01 mol %, or 0.001 mol %, based on the molar amount of the compound according to Formula (Ia).

In some embodiments, the method can occur in an inert atmosphere. In some embodiments, the method can occur in a N₂ atmosphere.

In some embodiments, the method further includes a solvent. The solvent can be any suitable solvent to one of ordinary skill in the art. In some embodiments, the solvent is an aprotic solvent. In some embodiments, the aprotic solvent can be aliphatic hydrocarbons, aromatic hydrocarbons, heteroaryls, ethers, halogenate hydrocarbons, or the like. For example, the aprotic solvent can include one or more of dichloromethane, 1,2 dichloroethane, tetrahydrofuran, ethyl acetate, diethyl ether, 1,4-dioxane, chloroform, pentane, hexane, benzene, chlorobenzene, toluene, pyridine, or the like. For example, the solvent can include one or more of dicholormethane, acetonitrile, and dimethylformamide.

The polymerization can occur at any temperature suitable to one of ordinary skill in the art. For example, the polymerization can occur at room temperature or a temperature of about −50° C. to about 100° C., or about −20° C. to about 80° C., or about 0° C. to about 60° C., or about −25° C. to about 30° C.

In some embodiments, the method further includes combining levoglucosan, a base, and a reactant to form the compound according to Formula (Ia). In some embodiments, the reactant can include a halide of R¹, such as, Cl—R¹, Br—R¹, I—R¹, wherein the halide is attached at the same point of attachment as the corresponding oxygen of the compound of Formula (I). The base can include any suitable base to one of ordinary skill in the art. For example, the base can be a hydroxide (e.g., NaOH, KOH, or the like), a hydride (e.g., NaH), or the like. In some embodiments, the method can further include a phase-transfer catalyst. The phase-transfer catalyst can be any suitable phase-transfer catalyst to one of ordinary skill in the art. For example, the phase-transfer catalyst can include tetrabutylammonium bromide, tetrabutylammonium hydroxide, or the like.

### Methods of Modifying a Compound According to Formula (I)

The present disclosure provides methods of modifying a polymer. In some embodiments, the methods include combining a thiol or an azide, and a compound according to Formula (I) as disclosed herein, to form a compound according to Formula (II):

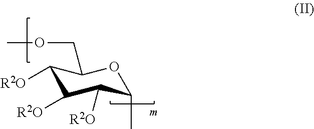

wherein:
each R² is independently C₂-C₂₀ alkyl, C₂-C₂₀ alkylene, C₂-C₂₀ alkylyne, C₁-C₂₀ heteroalkyl, C₁-C₈ heterocycloalkyl, C₂-C₂₀ heteroalkylene, C₂-C₂₀ heteroalkylyne, C₃-C₈ cycloalkyl, C₆-C₂₀ aryl, C₁-C₁₅ heteroaryl and each of the heteroalkyl, heterocycloalkyl, heteroalkylene, heteroalkylyne, and heteroaryl include 1-5 heteroatoms selected from N, O, and S;
at least one R² is a C₁-C₂₀ thioalkyl or C₁-C₁₅ heteroaryl; and
n and m are each independently in a range of 3 to 1000.

In some embodiments, at least one R is C₂-C₂₀ alkenyl or C₂-C₂₀ alkynyl. In some embodiments, each R is C₂-C₂₀ alkenyl or C₂-C₂₀ alkynyl.

In some embodiments, at least one R is C₂-C₂₀ alkenyl. In some embodiments, each R is C₂-C₂₀ alkenyl. In some embodiments, at least one R is

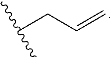 [SMILES: *CC=C]

In some embodiments, each R is

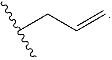 [SMILES: *CC=C]

In some embodiments, at least one R is C₂-C₂₀ alkynyl. In some embodiments, each R is C₂-C₂₀ alkynyl.

In some embodiments, at least one R² is a C₁-C₂₀ thioalkyl or C₁-C₁₅ heteroaryl. In some embodiments, each R² is a C₁-C₂₀ thioalkyl or C₁-C₁₅ heteroaryl.

In some embodiments, at least one R² is a C₂-C₂₀ thioalkyl. In some embodiments, each R² is a C₂-C₂₀ thioalkyl.

In some embodiments, at least one R² is

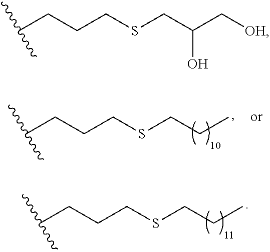

In some embodiments, each R² is

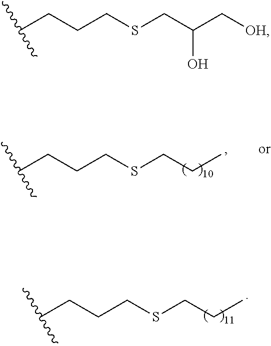

In some embodiments, at least one R² is C₁-C₁₅ heteroaryl. In some embodiments, each R² is C₁-C₁₅ heteroaryl. In some embodiments, the heteroaryl is triazolyl.

In some embodiments, each R² C₁-C₂₀ heteroalkyl, C₁-C₈ heterocycloalkyl, or C₁-C₁₅ heteroaryl and each of the heteroalkyl, heterocycloalkyl, and heteroaryl comprise 1-5 heteroatoms selected from N, O, and S.

In some embodiments, at least one R² is independently C₁-C₂₀ heteroalkyl, C₁-C₈ heterocycloalkyl, or C₁-C₁₅ heteroaryl. In some embodiments, each R² is independently C₁-C₂₀ heteroalkyl, C₁-C₈ heterocycloalkyl, or C₁-C₁₅ heteroaryl.

In some embodiments, at least one R² is

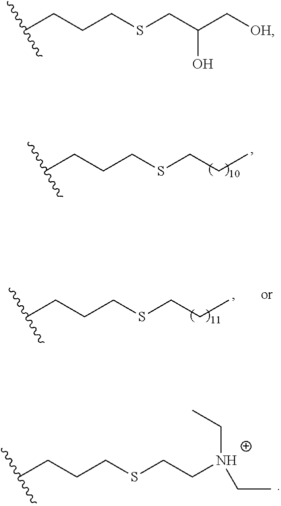

In some embodiments, each R² is

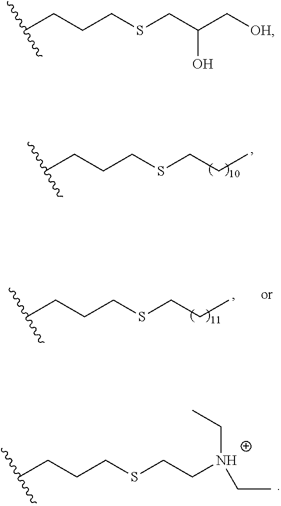

In some embodiments, R² is

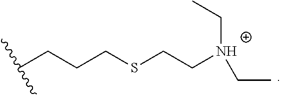 [SMILES: *CCCSCC[NH+](CC)CC]

In some embodiments, at least one R² is cationic. In some embodiments, each R² is cationic. In some embodiments, at least one R² comprises a cationic amine. For example, the cationic amine can include, but is not limited to, —(N(CH₃)₃)⁺, —(NH₃)⁺, —(NH(CH₂CH₃)₂), —(NH(CH₃)₂)⁺, and

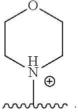

In some embodiments, a compound according to Formula (II) as disclosed herein is cationic.

In some embodiments, the thiol is a C₁-C₂₀ thiol. In some embodiments, the azide is a C₁-C₂₀ azide. In some embodiments, the thiol can be a mixture of 2 or more different thiols. In some embodiments, the azide can be a mixture of 2 or more different azides.

In some embodiments, the method further includes irradiating the mixture with ultra violet light. In some embodiments, the irradiating can occur for about 1 minute to about 1 hour, or about 1 minute to about 30 minutes, or about 10 minutes.

In some embodiments, the method further includes a radical polymerization initiator.

In some embodiments, the thiol or azide, and R² are present in a ratio of about 0.9:1 to about 1:3.

In some embodiments, m can in a range of 3 to 1000. For example, m can in a range of about 5 to about 500, or about 5 to about 400, or about 10 to about 250, or about 20 to about 150, or about 25 to about 100, or about 20 to about 75, or about 30 to 60.

The compounds according to Formula (I) or Formula (II) can be characterized by the number average molecular weight (Mₙ) or the weight average molecular weight (M_{w}). In some embodiments, the compounds according to Formula (II) can have a Mₙ in a range of about 1000 to about 500,000. For example, the compounds according to Formula (II) can have a Mₙ in a range of about 1000 to about 250,000, or about 1000 to about 150,000, or about 5000 to about 100,000, or about 2000 to about 20,000. In some embodiments, the compounds according to Formula (II) can have a Mₙ in a range of about 1000 to about 100,000.

In some embodiments, the compounds according to Formula (I) can have a Mₙ in a range of about 1000 to about 500,000. For example, the compounds according to Formula (I) can have a Mₙ in a range of about 1000 to about 250,000, or about 1000 to about 150,000, or about 5000 to about 100,000, or about 2000 to about 20,000. In some embodiments, the compounds according to Formula (I) can have a Mₙ in a range of about 1000 to about 100,000.

Some embodiments provide a pharmaceutical composition comprising a compound of Formula (I), or a pharmaceutically acceptable salt thereof, or a compound of Formula (II), or a pharmaceutically acceptable salt thereof and one or more pharmaceutically active ingredients (e.g., genetic therapeutics). In some embodiments, the one or more pharmaceutically active ingredients comprise antisense oligonucleotides (ASOs). In some embodiments, the one or more pharmaceutically active ingredients is an antisense oligonucleotide (ASO). Non-limiting examples of ASOs can include shRNA, siRNA, and morpholinos. In some embodiments, the one or more pharmaceutically active ingredients comprise mRNA.

Some embodiments provide methods of delivering one or more pharmaceutically active ingredients (e.g., an ASO) to a subject, the methodscomprising administering to the subject a therapeutically effective amount of the pharmaceutical composition as disclosed herein.

### Examples

Materials and Methods

Levoglucosan was purchased from Biosynth Carbosynth. 1,6-Anhydro-2,3,4-tri-O-benzyl-β-D-glucopyranose was obtained from Synthose Inc. Anhydrous dichloromethane (CH₂Cl₂, ≥99.8%) was purchased from Sigma and used without further purification for polymerizations. MeCN purchased from Fisher was dried over CaH₂ for 3 days, followed by three freeze-pump-thaw cycles, and vacuum transferred before use. Photoinitiator Omnirad 2100 (mixture of 90-95% ethyl(2,4,6-trimethylbenzoyl)phenylphosphinate and 5-10% phenylbis(2,4,6-trimethylbenzoyl)phosphine oxide) was generously provided by IGM Resins. Dextran (number average molecular weight (Mₙ of 18700 Da) was purchased from Biosynth Carbosynth. Bismuth(III) subsalicylate (99.9% trace metals basis, catalog #480789), diphenyl phosphoric acid (99%, catalog #850608), boron trifluoride diethyl etherate (catalog #175501), methyl trifluoromethanesulfonate (≥98%, catalog #164283), scandium(III) triflate (99%, catalog #418218), iron(III) trifluoromethanesulfonate (90%, catalog #708801), zinc trifluoromethanesulfonate (98%, catalog #290068), and aluminum isopropoxide (≥98%, catalog #220418) were purchased from Sigma Aldrich. Yttrium(III) Trifluoromethanesulfonate (≥98%, catalog #T19215G), lanthanum(III) trifluoromethanesulfonate (99%, catalog #AC343910010), aluminum trifluoromethanesulfonate (99%, catalog #AAB2078506), and titanium(IV) isopropoxide (≥98%, catalog #AC194700050) were purchased from Fisher Scientific. Bismuth(III) trifluoromethanesulfonate (99%, catalog #L19687) was purchased from Alfa Aesar. All solid polymerization catalysts were dried under vacuum for 24 h before use. All other reagents and solvents were purchased from commercial sources and used without additional purification.

Nuclear Magnetic Resonance (NMR): A Bruker Avance III HD 400 spectrometer at the University of Minnesota Twin Cities was used to obtain all ¹H and ¹³C NMR spectrums except for the ¹H NMR spectrum of poly(4). For better resolution the ¹H NMR spectrum of poly(4) was obtained on the Bruker Avance III 500 MHz spectrometer at the University of Minnesota Twin Cities. ¹H NMR spectra were referenced to residual solvent shifts of CHCl₃=7.26 ppm or CH₃OH=3.31 ppm, and ¹³C NMR spectra were referenced to residual solvent shifts of CHCl₃=77.16 ppm. All NMR spectra were analyzed using MestReNova.

Mass Spectrometry: High-resolution mass spectra (HRMS) of synthesized monomer was collected on a Bruker BioTOF II (ESI-TOF) instrument in electrospray ionization (ESI) mode using PPG as an internal calibrant/standard on an Agilent 7200 GC/QTOF instrument. The sample was dissolved in methanol.

Glovebox: All polymerizations were carried out under nitrogen atmosphere in an MBraun Labmaster glovebox.

Size Exclusion Chromatography (SEC): Molecular weight (Mₙ and M_{w}) and dispersity (M_{w}/Mₙ) of poly(2), poly(3), and poly(4) were determined in DMF containing 0.05M LiBr at a flow rate of 1 mL per minute using an Agilent Infinity 1260 HPLC. The SEC was equipped with a Wyatt DAWN Heleos II multiangle laser light scattering detector (3 angles from 100 to 180°) and a Wyatt OPTILAB T-rEX refractive index detector. dn/dc was calculated from the refractive index signal using a known sample concentration and assuming 100% mass recovery.

Mₙ, M_{w}, and M_{w}/Mₙ of poly(5) was determined in THF with a flow rate of 1 mL per minute on an Agilent Infinity 1260 HPLC with Waters Styragel (HR6, HR5, and HR1) columns connected to a Wyatt DAWN Heleos II multiangle laser light scattering detector (18 angles from 100 to 180°) and a Wyatt OPTILAB T-rEX refractive index detector. dn/dc was calculated from the refractive index signal using a known sample concentration and assuming 100% mass recovery.

Thermogravimetric Analysis (TGA): TGA analyses were performed on a TA Instruments Q500 at a heating rate of 10° C. min⁻¹ under nitrogen atmosphere.

Differential Scanning Calorimetry (DSC): Thermal properties of the polymers were characterized using the Mettler Toledo DSC 1 instrument under nitrogen atmosphere.

Real Time-Fourier Transform Infrared (RT-FTIR): The thiol-ene post polymerization modification kinetics of poly(3) were studied via RT-FTIR using a Thermo Fisher Scientific Nicolet 6700 FTIR spectrometer. A drop of the freshly prepared mixture containing poly(3), thiol and Omnirad 2100 was sandwiched between two polished NaCl plates. Once the sample was exposed to UV light, spectra were recorded every 20 ms at an average of 1 scan with a spectral resolution of 16 cm⁻¹. To calculate poly(3) ene conversion due to UV irradiation (OmniCure S1500 Curing System, 100 mW/cm²), the intensity reduction of C═C double bond peak at 925 cm⁻¹ as compared to the initial peak was calculated. The double bond peak area was normalized to the non-reactive CH₂ stretches at 2976 cm⁻¹ for the thioglycerol mixture, and 2926 cm⁻¹ for the lauryl mercaptan mixture respectively.

Optical Rotation: The optical rotation was measured at 20° C. in CHCl₃ via the Rudolph Research Analytical Autopol III Automatic Polarimeter. The measurement was performed in triplicate.

Monomer 2 was purchased from a commercial supplier.

### Example 1: Synthesis of Monomer 3

Synthetic Route 1

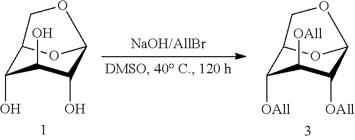

As used herein, the abbreviation “All” stands for allyl

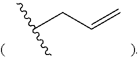 [SMILES: *CC=C]

In a 250 mL round-bottom flask, levoglucosan, 1, (5 g, 30.84 mmol, 1 equiv.) was dissolved in dimethyl sulfoxide (30.84 mL) by stirring at room temperature. Sodium hydroxide pellets (4.07 g, 101.8 mmol, 3.3 equiv.) were ground into a fine powder. Freshly powdered sodium hydroxide was added to the levoglucosan solution while stirring at the rate of 400 rpm. The solution was heated to 40° C. and allyl bromide (8.81 mL, 101.8 mmol, 3.3 equiv.) was added dropwise over the course of 15 min. One additional aliquot of powdered sodium hydroxide (3.3 equiv.) and allyl bromide (3.3 equiv.) was added to the solution at the 24 h and 48 h time points.

After 120-hour reaction, the aqueous layer was extracted with diethyl ether (3×200 mL). The organic layer was dried over Na₂SO₄ and the filtrate was then concentrated via rotary evaporation. The crude mixture was subjected to column chromatography on silica gel (4:1 Hexanes:EtOAc) to isolate monomer 3 as an oil (Yield=75%).

Synthetic Route 2

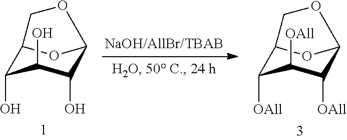

Disclosed herein is a “green” synthetic pathway for monomer 3 using NaOH, a phase-transfer catalyst, and water as the reaction medium. The monomer was successfully prepared with this method below.

In a 50 mL round-bottom flask, levoglucosan, 1, (2 g, 12.34 mmol, 1 equiv.) and the phase transfer catalyst tetrabutylammonium bromide (TBAB, 240 mg, 0.74 mmol, 0.06 equiv.) were weighed out. Sodium hydroxide pellets (2.97 g, 74.04 mmol, 6 equiv.) were dissolved in DI water (11.88 ml) to obtain a 25% NaOH aqueous solution. The levoglucosan-TBAB mixtures was dissolved in the 25% NaOH aqueous solution by stirring at room temperature. The solution was heated to 50° C. and allyl bromide (6.5 ml, 74.04 mmol, 6 equiv.) was added dropwise over the course of 15 min.

After 24-hour reaction, the aqueous layer was extracted with dichloromethane (3×100 mL). The organic layer was dried over Na₂SO₄ and the filtrate was then concentrated via rotary evaporation. The crude mixture was subjected to column chromatography on silica gel (4:1 Hexanes:EtOAc) to isolate monomer 3 as an oil (Yield=36%).

¹H NMR (400 MHz, CDCl₃): δ 5.98-5.84 (m, 3H, —O—CH₂—CH═CH₂), 5.43 (s, 1H, —O—CH—O), 5.34-5.16 (m, 6H, —O—CH₂—CH═CH₂), 4.57 (d, J=8 Hz, 1H), 4.17-4.07 (m, 6H, —O—CH₂—CH═CH₂), 3.92 (d, J=4 Hz, 1H), 3.70 (t, J=6 Hz, 1H), 3.51 (s, 1H), 3.30 (s, 1H), 3.27 (s, 1H).

¹³C NMR (400 MHz, CDCl₃): δ 134.81, 134.7, 134.67, 117.76, 117.62, 117.33, 100.81, 77.36, 74.88, 71.42, 71.21, 70.64, 65.69.

HRMS (ESI-TOF): [3+Na]+: calculated 305.1359; found 305.1361.

### Example 2: Catalyst Screening and Solvent Screening

### Comparative Example—Polymerization of Monomer 2

Synthetic Route 1: In a nitrogen-filled glovebox, the required amount of catalyst (e.g., 0.01 mmol for 2 mol % catalyst loading) was weighed out in an 8 ml scintillation vial. A Teflon coated stir bar was added to this vial. Monomer 2 (0.5 mmol) was dissolved in CH₂Cl₂ or MeCN (e.g. 500 μL for initial monomer concentration of 1M) and added to the vial containing the catalyst. The vial was sealed with a Teflon lined cap and the polymerization solution was stirred at RT inside the glovebox. After 72 hours, the vial was removed from the glovebox. The polymerization was quenched with methanol (100 μL) or tert-butyl alcohol (100 μL) and stirred for an additional 10 mins. The crude reaction mixture was analyzed by ¹H NMR spectroscopy to determine conversion. The polymer was then precipitated twice into cold methanol. The methanol was removed, and the polymer was dried under vacuum overnight. Typical poly(2) yield ranged from 65 to 90%.

Synthetic Route 2: In a nitrogen-filled glovebox, the required amount of Sc(OTf)₃ (e.g., 0.01 mmol for 2 mol % catalyst loading) was weighed out in an 8 ml scintillation vial. MeCN (e.g., 50 μL for 10% MeCN) was added to the vial containing Sc(OTf)₃, along with a Teflon coated stir bar. Monomer 2 (0.5 mmol) was dissolved in CH₂Cl₂ (e.g., 450 μL for 90% CH₂Cl₂ and for total initial monomer concentration of 1M) and added to the vial containing Sc(OTf)₃. The vial was sealed with a Teflon lined cap and the polymerization solution was stirred at RT inside the glovebox. After 72 hours, the vial was removed from the glovebox. The polymerization was quenched with methanol (100 μL) or tert-butyl alcohol (100 μL) and stirred for an additional 10 minutes. The crude reaction mixture was analyzed by ¹H NMR spectroscopy to determine conversion. The polymer was then precipitated twice into cold methanol. The methanol was removed, and the polymer was dried under vacuum overnight. Typical poly(2) yield ranged from 37 to 68%.

Monomer conversion was determined by ¹H NMR analysis of the crude polymerization mixture. For this purpose, the C₁ anomeric proton was used for tracking conversion. The conversion was calculated as follows. H, corresponds to the integration of polymer repeat unit proton and H_{M} corresponds to the integration of the anomeric proton in monomer 2.

$$\int {H}_{P} = \frac{26.96 - 7}{7} = 2.85$$

$$Conversion = \frac{\int {H}_{P}}{\int {H}_{P} + \int {H}_{M}} = \frac{2.85}{2.85 + 1} = 0.74$$

Polymerization of Monomer 3

Synthetic Route 1: In a nitrogen-filled glovebox, the required amount of catalyst (e.g., 0.017 mmol for 5 mol % catalyst loading) was weighed out in an 8 ml scintillation vial. A Teflon coated stir bar was added to this vial. Monomer 3 (0.354 mmol) was dissolved in CH₂Cl₂ (354 μL for initial monomer concentration of 1M) and added to the vial containing the catalyst. The vial was sealed with a Teflon lined cap and the polymerization solution was stirred at RT inside the glovebox. After 72 hours, the vial was removed from the glovebox.

The polymerization was quenched with tert-butyl alcohol (100 μL) and stirred for an additional 10 minutes. The crude reaction mixture was analyzed by ¹H NMR spectroscopy to determine conversion. The polymer was precipitated into a cold mixture of 50:50 water: methanol. The precipitation solvent was then removed, and the isolated polymer was redissolved in CH₂Cl₂. Water was removed by drying over Na₂SO₄, followed by rotary evaporation of the filtrate and finally drying the polymer under vacuum overnight. Typical poly(3) yield ranged from 38 to 91%.

Synthetic Route 2: In a nitrogen-filled glovebox, the required amount of Bi(OTf)₃ (e.g., 0.017 mmol for 5 mol % catalyst loading) was weighed out in an 8 ml scintillation vial. MeCN (e.g., 35.4 μL for 10% MeCN) was added to the vial containing Sc(OTf)₃, along with a Teflon coated stir bar. Monomer 3 (0.354 mmol) was dissolved in CH₂Cl₂ (318.6 μL for 90% CH₂Cl₂ and for initial monomer concentration of 1M) and added to the vial containing Bi(OTf)₃. The vial was sealed with a Teflon lined cap and the polymerization solution was stirred at RT inside the glovebox. After 72 hours, the vial was removed from the glovebox. The polymerization was quenched with tert-butyl alcohol (100 μL) and stirred for an additional 10 minutes. The crude reaction mixture was analyzed by ¹H NMR spectroscopy to determine conversion. The polymer was precipitated into a cold mixture of 50:50 water: methanol. The precipitation solvent was then removed, and the isolated polymer was redissolved in CH₂Cl₂. Water was removed by drying over Na₂SO₄, followed by rotary evaporation of the filtrate and finally drying the polymer under vacuum overnight. Typical poly(3) yield ranged from 53 to 87%.

Monomer conversion was determined by ¹H NMR analysis of the crude polymerization mixture. For this purpose, the C₁ anomeric proton was used to track the conversion. The conversion was calculated as follows. H, corresponds to the integration of polymer repeat unit proton and H_{M} corresponds to the integration of the anomeric proton in monomer 3.

$$\int {H}_{P} = \frac{46.48 - 6}{6} = 6.75$$

$$Conversion = \frac{\int {H}_{P}}{\int {H}_{P} + \int {H}_{M}} = \frac{6.75}{6.75 + 1} = 0.86$$

To identify less toxic catalysts to promote cROP of monomer 2 and 3, and to understand the effect of solvent and catalyst loading on cROP of monomer 2 and 3, a library of polymerization experiments was performed. A range of metal and organic catalysts was initially screened due to their commercial availability, low toxicity, and ability to ring open cyclic ethers for the polymerization of monomer 2 (Table 1). All screening reactions took place at room temperature for 72 h in dichloromethane (initial monomer concentration [M]₀=1.0 mol L⁻¹). Successful catalysts were then used to screen cROP of monomer 3; two metal triflates [Sc(OTf)₃ and Bi(OTf)₃] were identified for cROP of both monomers 2 and 3 (FIG. 3, entries 1, 2, 7, 8). Both metal triflates provided comparable conversion to the BF₃OEt₂ and MeOTf controls (Table 2) and provide the additional benefit of being recyclable (via a simple aqueous extraction) while not releasing corrosive byproducts such as triflic acid commonly released by alkyl triflates. However, these metal triflates have limited solubility in dichloromethane and hence further studies were performed to understand solvent effects.

The summary of the cROP catalyst screening for monomer 2 and 3 is shown in Table 1 below. The “O” indicates a non-zero conversion, “X” indicates zero conversion, and “NA” indicates not applicable.

TABLE 1

| Catalyst | 2a | 3b |
|---|---|---|
| Bismuth subsalicylate | X | NA |
| Diphenyl phosphoric acid | X | NA |
| Ti(iOPr)₄ | X | NA |
| Al(iOPr)₃ | X | NA |
| BF₃OEt₂ | O | X |
| MeOTf | O | O |
| Fe(OTf)₃ | X | NA |
| Sc(OTf)₃ | O | O |
| Al(OTf)₃ | X | NA |
| Bi(OTf)₃ | O | O |
| La(OTf)₃ | X | NA |
| Y(OTf)₃ | X | NA |
| Zn(OTf)₃ | X | NA |

aPolymerization conditions for 2: [2]₀ = 1M in CH₂Cl₂ at 25° C., 2:Cat = 50:1, time = 72 h.
bPolymerization conditions for 3: [3]₀ = 1M in CH₂Cl₂ at 25° C., 3:Cat = 20:1, time = 72 h.

The results of the catalyst screening that were marked showed non-zero conversion are shown below. Polymerizations were performed for 72 h in CH₂Cl₂ at RT, [2]₀=1M, and [3]₀=1M.

TABLE 2

| | | Monomer: | Conversion^{a} | Mₙ^{b} | M_{w}^{b} | |
|---|---|---|---|---|---|---|
| Monomer | Catalyst | Catalyst | (%) | (g/mol) | (g/mol) | Ð^{b} |
| 2 | BF₃OEt₂ | 50:1 | 66 | 8700 | 14500 | 1.7 |
| 2 | MeOTf | 50:1 | 70 | 4300 | 6300 | 1.5 |
| 2 | Sc(OTf)₃ | 50:1 | 74 | 6700 | 11030 | 1.6 |
| 2 | Bi(OTf)₃ | 50:1 | 84 | 3500 | 4400 | 1.3 |
| 3 | MeOTf | 20:1 | 92 | 3600 | 9100 | 2.5 |
| 3 | Sc(OTf)₃ | 20:1 | 77 | 3500 | 7900 | 2.3 |
| 3 | Bi(OTf)₃ | 20:1 | 86 | 3600 | 6100 | 1.7 |

^{a}Monomer conversion by ¹H NMR spectroscopy.
^{b}Molecular weights and dispersity determined by SEC-MALS in DMF

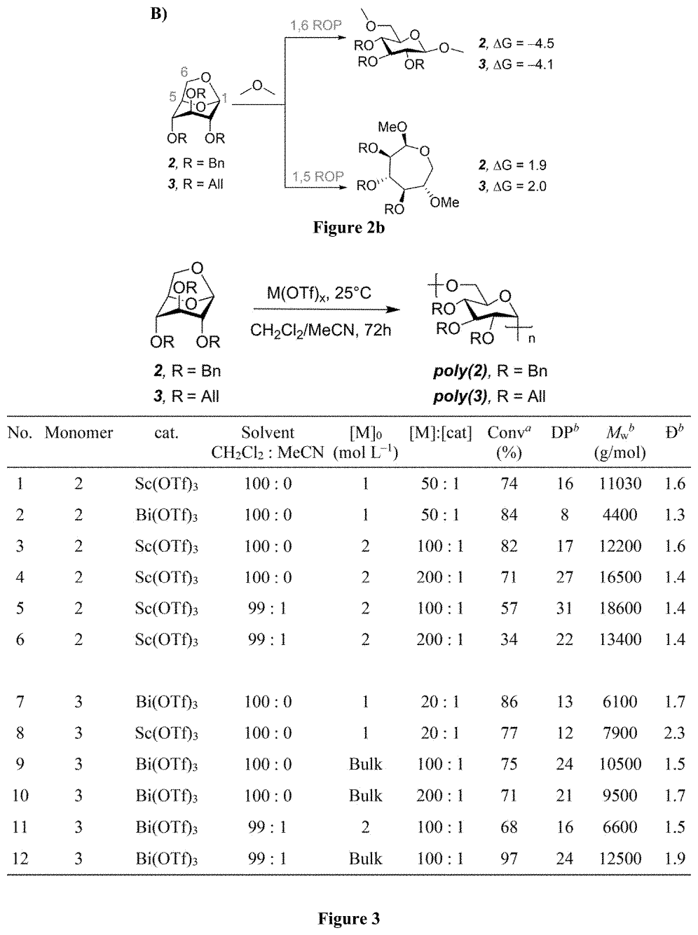

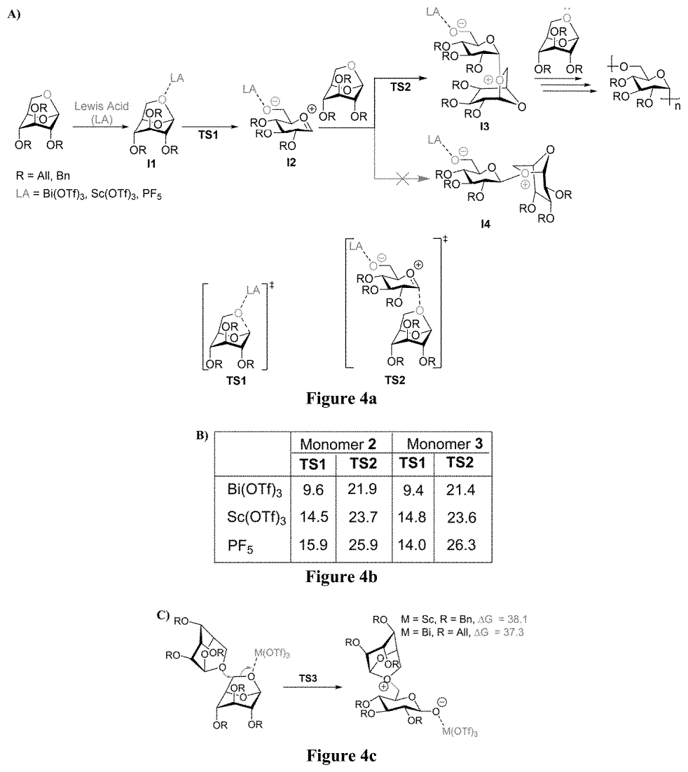

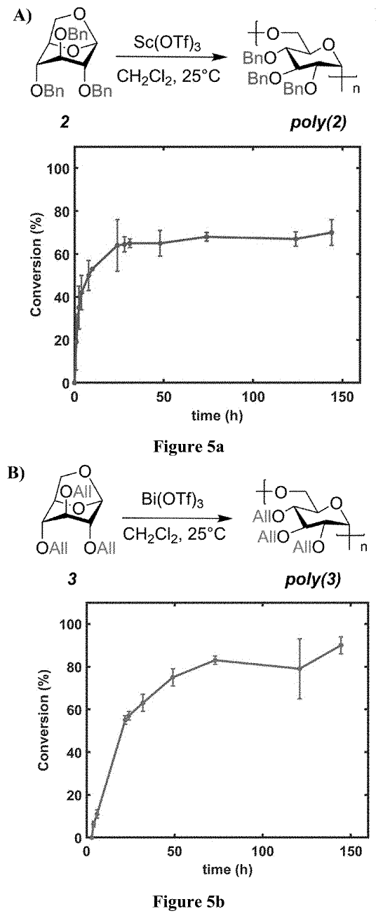

Further investigated was the addition of varying quantities of acetonitrile (MeCN) in the polymerization mixture. Interestingly, no polymerization occurred when >1000 MeCN by volume was present in the reaction medium. Moreover, monomer conversion generally decreased with increasing amount of MeCN from 0-10% (Tables 3 and 4). In addition, the effect of varying catalyst loading was studied on cROP of monomers 2 and 3 and found that polymerization of both monomers could be conducted at M(OTf)₃ loadings as low as 0.5 mol % (FIG. 3, entries 4, 6, 10). Notably, the pendant groups do not seem to drastically impact conversion, as similar values were observed for both the monomers likely due to their comparable ring-strain (FIG. 2b). With the identified metal triflates, Mw values up to 18.6 kDa for poly(2) (DP=31) and 12.5 kDa for poly(3) (DP=24) were achieved with moderate dispersities (FIG. 3, entries 5 and 12). These moderate molecular weights were likely caused by intermolecular chain transfer and back-biting reactions which are common features in cROP of cyclic acetals. Since the metal triflate mediated cROP of levoglucosan follows a catalytic approach, calculating a desired or target molecular weight for a given set of polymerization conditions is difficult.

The summary of the cROP solvent screen for monomers 2 and 3 is shown below in Table 3. The “O” indicates a non-zero conversion, “X” indicates zero conversion, and “NA” indicates not applicable.

TABLE 3

| Solvent CH₂Cl₂:MeCN | 2ª | 3^{b} |
|---|---|---|
| 100:0 | O | O |
| 0:100 | X | NA |
| 50:50 | X | NA |
| 80:20 | X | NA |
| 90:10 | O | O |
| 99:1 | O | O |

^{a}Polymerization conditions for 2: [2]₀ = 1M at 25° C., 2:Sc(OTf)₃ = 50:1, time = 72 h.
^{b}Polymerization conditions for 3: [3]₀ = 1M at 25° C., 3:Bi(OTf)₃ = 20:1, time = 72 h.

The results of the solvent systems that showed non-zero conversion is shown in Table 4 below. Polymerizations were performed for 72 h at RT, [2]₀=1M, and [3]₀=1M.

TABLE 4

| | | Solvent | Monomer: | Conversion^{a} | Mₙ^{b} | M_{w}^{b} | |
|---|---|---|---|---|---|---|---|
| Monomer | Catalyst | CH₂Cl₂:MeCN | Catalyst | (%) | (g/mol) | (g/mol) | Ð^{b} |
| 2 | Sc(OTf)₃ | 100:0 | 50:1 | 74 | 6700 | 11030 | 1.6 |
| 2 | Sc(OTf)₃ | 99:1 | 50:1 | 66 | 8800 | 12500 | 1.43 |
| 2 | Sc(OTf)₃ | 90:10 | 50:1 | 58 | 9630 | 12700 | 1.32 |
| 3 | Bi(OTf)₃ | 100:0 | 20:1 | 86 | 3600 | 6100 | 1.7 |
| 3 | Bi(OTf)₃ | 99:1 | 20:1 | 89 | 3400 | 5020 | 1.6 |
| 3 | Bi(OTf)₃ | 90:10 | 20:1 | 74 | 2300 | 3000 | 1.3 |

^{a}Monomer conversion by ¹H NMR spectroscopy.
^{b}Molecular weights and dispersity determined by SEC-MALS in DMF

Additional results of varying catalyst loading and initial monomer concentration on cROP of monomer 2 are shown below in Table 5.

TABLE 5

| Solvent | Monomer | | | | | |
|---|---|---|---|---|---|---|
| CH₂Cl₂: | Concentration | Monomer: | Conversion^{a} | Mₙ^{b} | M_{w}^{b} | |
| MeCN | (M) | Catalyst | (%) | (g/mol) | (g/mol) | Ð^{b} |
| 100:0 | 1 | 100:1 | 68 | 4100 | 8530 | 2.1 |
| 100:0 | 1 | 333:1 | 0 | — | — | — |
| 99:1 | 1 | 100:1 | 28 | 7100 | 9060 | 1.3 |
| 99:1 | 2 | 1000:1 | 0 | — | — | — |

^{a}Monomer conversion by ¹H NMR spectroscopy.
^{b}Molecular weights and dispersity determined by SEC-MALS in DMF. Polymerization conditions: Sc(OTf)₃ catalyst, 72 h, RT.

¹H NMR analysis of poly(2) and poly(3) compared to the respective monomers indicates that the polymers possess 1,6-α glycosidic stereoregularity as the anomeric proton resonances (in the β-configuration) appeared at 4.94 ppm (Kakuchi et al., Cationic ring-opening polymerization of 1,6-anhydro-2,3,4-tri-O-allyl-b-D-glucopyranose as a convenient synthesis of dextran, Macromol. Rapid Commun., 2000, 21, 1003-1006). Additionally ¹³C NMR of poly(2) and poly(3) also showed the α configuration as the anomeric carbon resonances appeared at 97.8 ppm and 97.4 ppm, respectively (Fu et al., Synthesis of clickable amphiphilic polysaccharides as nanoscopic assemblies, Chem. Commun., 2014, 50, 12742-12745). The stereoregularity of the polymers was also confirmed via optical rotation measurements. The optical rotation values for poly(2) and poly(3) were measured to be +91.5° cm³g⁻¹dm⁻¹ and +84° cm³g⁻¹dm⁻¹ respectively, with the optical rotation values for monomers 2 and 3 being −31.5° cm³g⁻¹dm⁻¹ and −45° cm³g⁻¹dm⁻¹, respectively. Overall, these results indicate that the cROP of monomers 2 and 3 with metal triflates yields highly stereoregular levoglucosan polymers with 1,6-α glycosidic linkages. Notably, this disclosure demonstrates that highly stereoregular levoglucosan polymers were synthesized under mild conditions without the need for an energy intensive process involving high vacuum and low temperatures. ¹H NMR analysis also indicates that the alkene functionality in poly(3) is intact during cROP with the vinyl proton resonances at 5.91 ppm, 5.27 ppm and 5.14 ppm. The successful synthesis of poly(3) while preserving multiple pendant allyl groups allowed for further click-chemistry modifications.

### Example 3: Polymerization Kinetics

Overall cROP kinetics of Comparative Monomer 2—In a nitrogen-filled glovebox, 4.27 mg of Sc(OTf)₃ (0.009 mmol for 0.5 mol % catalyst loading) was weighed out in an 8 ml scintillation vial. A Teflon coated stir bar was added to this vial. 750 mg of monomer 2 (1.74 mmol) was dissolved in CH₂Cl₂ (870 μL for initial monomer concentration of 2M) and added to the vial containing the catalyst. The vial was sealed with a Teflon lined cap and the polymerization solution was stirred at RT inside the glovebox. At each specified time point, an aliquot was taken from this reaction mixture inside the glovebox and was quenched by adding methanol. The crude reaction mixture was analyzed by ¹H NMR spectroscopy to determine conversion. This study was done in triplicate shown in Table 6 below.

TABLE 6

| | Conversion | Conversion | Conversion |
|---|---|---|---|
| Time | (%) | (%) | (%) |
| point (h) | Replicate 1 | Replicate 2 | Replicate 3 |
| 1 | 19 | 24 | 32 |
| 2.5 | 35 | 45 | 37 |
| 4 | 42 | 50 | 43 |
| 8 | 50 | 57 | 53 |
| 10 | 53 | 53 | 53 |
| 24 | 64 | 68 | 56 |
| 28 | 64 | 63 | 61 |
| 31 | 65 | 67 | 66 |
| 48 | 65 | 66 | 60 |
| 74 | 68 | 70 | 69 |
| 124 | 67 | 70 | 68 |
| 144 | 70 | 76 | 72 |

Determination of rate law for cROP of monomer 2—Based on the results of the previous study, it was decided that conversion data between 0 and 4 h will be utilized to evaluate the reaction order for cROP of monomer 2. To obtain sufficient time points within this region, the kinetic study was repeated in duplicate for a total reaction time of 3 hours. In a nitrogen-filled glovebox, 5.13 mg of Sc(OTf)₃ (0.0104 mmol for 0.5 mol % catalyst loading) was weighed out in an 8 ml scintillation vial. A Teflon coated stir bar was added to this vial. 900 mg of monomer 2 (2.08 mmol) was dissolved in CH₂Cl₂ (1040 μL for initial monomer concentration of 2M) and added to the vial containing the catalyst. The vial was sealed with a Teflon lined cap and the polymerization solution was stirred at RT inside the glovebox. At each specified time point, an aliquot was taken from this reaction mixture inside the glovebox and was quenched by adding isopropanol. The crude reaction mixture was analyzed by ¹H NMR spectroscopy to determine conversion. The results are shown in Table 7 below and FIG. 7.

For cROP of monomer 2, k_{obs} could not be calculated and it was hypothesized that the cROP kinetics for monomer 2 may follow more complex rate laws.

TABLE 7

| Time point | Conversion (%) | Conversion (%) |
|---|---|---|
| (min) | Replicate 1 | Replicate 2 |
| 0 | 0 | 0 |
| 5 | 0 | 0 |
| 20 | 5 | 8 |
| 30 | 12 | 17 |
| 45 | 17 | 28 |
| 65 | 29 | 33 |
| 75 | 33 | 38 |
| 90 | 33 | 40 |
| 105 | 37 | 42 |
| 120 | 38 | 43 |
| 135 | 38 | 46 |
| 150 | 44 | 47 |
| 165 | 47 | 51 |
| 180 | 44 | 44 |

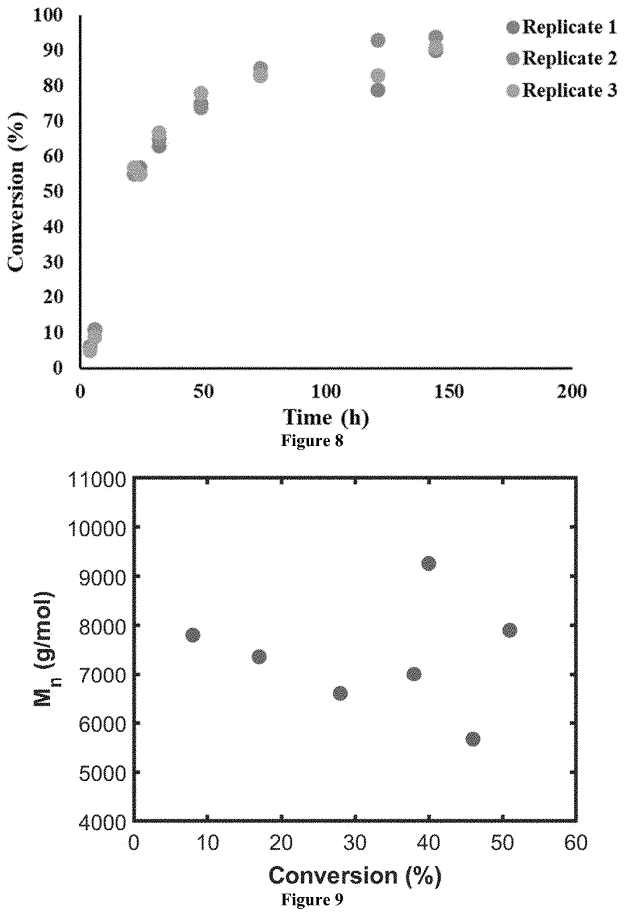

Overall cROP kinetics of monomer 3—In a nitrogen-filled glovebox, 28 mg of Bi(OTf)₃ (0.043 mmol for 1 mol % catalyst loading) was weighed out in an 8 ml scintillation vial. A Teflon coated stir bar was added to this vial. 1.2 g of monomer 3 (4.27 mmol) was dissolved in CH₂Cl₂ (610 μL for initial monomer concentration of 7M) and added to the vial containing the catalyst. The vial was sealed with a Teflon lined cap and the polymerization solution was stirred at RT inside the glovebox. At each specified time point, an aliquot was taken from this reaction mixture inside the glovebox and was quenched by adding isopropanol. The crude reaction mixture was analyzed by ¹H NMR spectroscopy to determine conversion. This study was done in triplicate and the data is shown in Table 8 and shown in FIG. 8.

The filling results for monomer 3 indicated a second order rate law with respect to monomer concentration and a k_{obs} of 0.0104 M⁻¹ h⁻¹ was determined for this monomer.

TABLE 8

| | | Conversion | Conversion | Conversion |
|---|---|---|---|---|
| Time point | | (%) | (%) | (%) |
| (min or h) | | Replicate 1 | Replicate 2 | Replicate 3 |
| 0 | min | 0 | 0 | 0 |
| 15 | min | 0 | 0 | 0 |
| 30 | min | 0 | 0 | 0 |
| 45 | min | 0 | 0 | 0 |
| 60 | min | 0 | 0 | 0 |
| 75 | min | 0 | 0 | 0 |
| 90 | min | 0 | 0 | 0 |
| 105 | min | 0 | 0 | 0 |
| 120 | min | 0 | 0 | 0 |
| 180 | min | 0 | 0 | 0 |
| 4 | h | 6.3 | 6.3 | 5 |
| 6 | h | 11 | 11 | 9 |
| 22 | h | 55 | 55 | 57 |
| 24 | h | 57 | 55 | 55 |
| 32 | h | 63 | 65 | 67 |
| 49 | h | 75 | 74 | 78 |
| 73 | h | 83 | 85 | 83 |
| 121 | h | 79 | 93 | 83 |
| 144.5 | h | 90 | 94 | 91 |

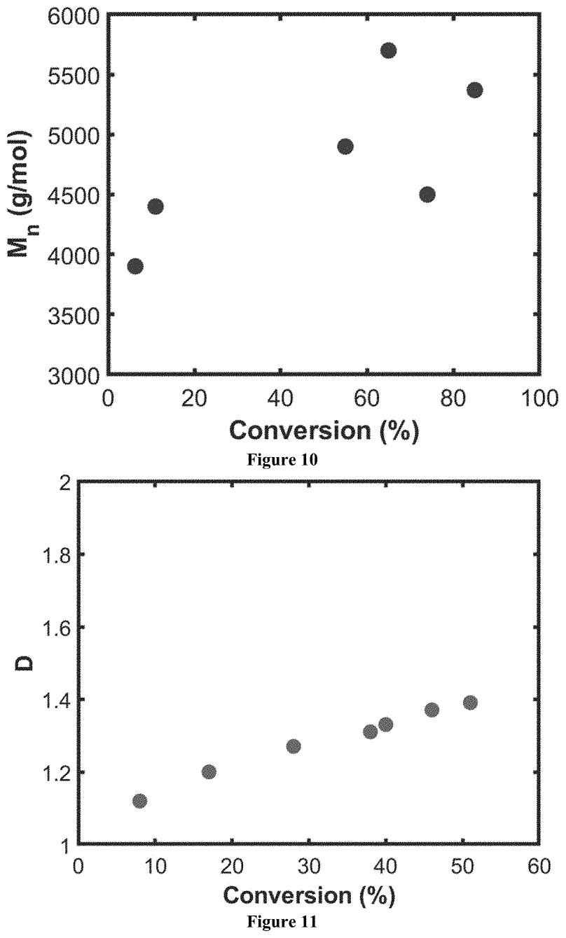

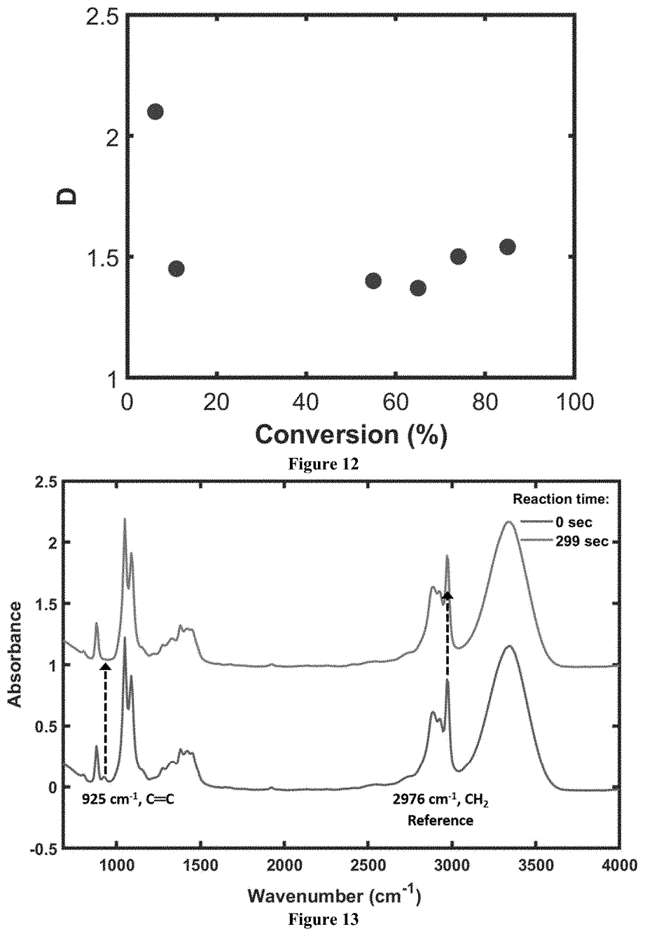

The molecular weight versus conversion for cROP kinetics of monomer 2 and 3 was compared and is shown in FIGS. 9-12. FIG. 9 shows the Mₙ versus conversion for cROP of monomer 2. FIG. 10 shows the Mₙ versus conversion for cROP of monomer 3. FIG. 11 shows the D versus conversion for cROP of monomer 2. FIG. 12 shows the D versus conversion for cROP of monomer 3.

A short induction period of ˜20 min was observed during cROP of monomer 2 (Table 7), which reaches an equilibrium conversion of 64% in ≤24 h (FIG. 5a). Conversely, cROP of monomer 3 has a much longer induction period of ˜4 h (Table 8) and reaches an equilibrium conversion of 83% in ≤72 h (FIG. 5b). Remarkably, the induction period for cROP of monomer 3, as evidenced by ¹H NMR spectroscopy, was accompanied with a drastic color change in the solution throughout the 4 h period. It is hypothesized long cROP induction period of monomer 3 was due to the non-productive coordination of Bi(OTf)₃ with the allylic ether oxygens and the glucopyranose ring oxygen. This was supported by DFT energetics calculations depicting that non-productive coordination of Bi with the allylic ether oxygens and the glucopyranose ring oxygen is favored at multiple locations (FIG. 5c). Furthermore, while comparing monomers 2 and 3, the relative free energies of binding the M(OTf)_{X} to each of these respective oxygen atoms vary slightly (FIG. 5c). However, the free energy trends and magnitudes are similar across both monomers 2 and 3. Along with tracking conversion over time, the molar mass and dispersity of the growing polymer chains was also monitored throughout this kinetic study. For poly(2), the Mₙ increased up to 40% conversion (FIG. 9), and for poly(3) the Mₙ increased up to 60% conversion (FIG. 10). Lastly, narrow dispersities were maintained for poly(2) throughout the reaction duration (˜1.2 to 1.4, FIG. 11), whereas for poly(3) dispersity was higher in the initial stages and gradually decreased to a stable value (˜1.5, FIG. 12).

### Example 4: Post Polymerization Modification of poly(3) with Thioglycerol

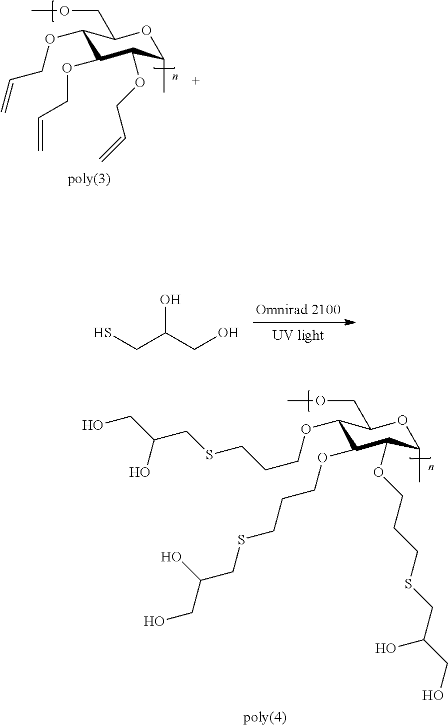

In a 4 mL vial, poly(3) (100 mg, Mₙ=6760 Da, fₑₙₑ=72, 0.015 mmol, 1 equiv.) was dissolved in ethanol (750 μL) by vortexing at room temperature. In a separate 4 mL vial, Omnirad 2100 (19.6 mg, 9 wt % of total formulation weight) and thioglycerol (117.5 mg, fₜₕᵢₒₗ=1, 1.08 mmoles, 72 equiv.) were weighed out to maintain an alkene:thiol of 1:1. The solution of poly(3) in ethanol was then added to the vial containing thioglycerol and further mixed by vortexing at room temperature. The formulation was irradiated with UV light (OmniCure S1500 Curing System, 100 mW/cm²) for 10 min to synthesize poly(4).

The synthesized polymer, poly(4), was isolated from the crude mixture via a two-step purification method. In the first step, the crude mixture was redissolved in excess water and extracted three times with dichloromethane to remove unreacted poly(3). The aqueous layer was then dialyzed in water (1 kDa RC dialysis tubing) to remove any unreacted thioglycerol.

The purified polymer was concentrated by rotary evaporation and finally the polymer was dried under vacuum for 48 h. The poly(4) was analyzed by RT-FTIR and the FTIR spectra is shown in FIG. 13.

### Example 5: Post Polymerization Modification of poly(3) with Lauryl Mercaptan

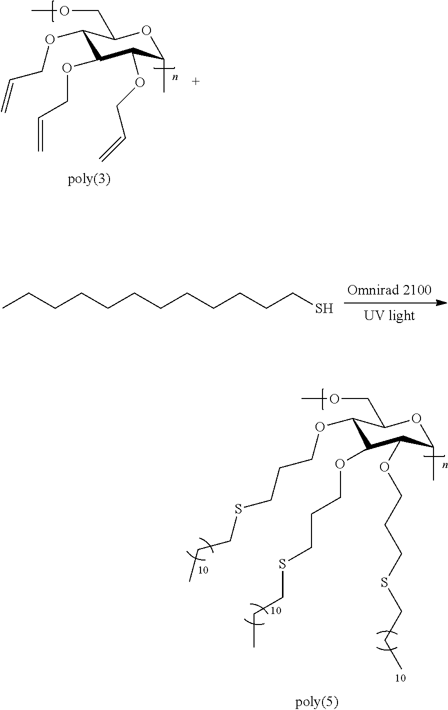

In a 4 mL vial, poly(3) (100 mg, M_{w}=6760 Da, fₑₙₑ=72, 0.015 mmole 1 equiv.) was dissolved in dichloromethane (750 μL) by vortexing at room temperature. In a separate 4 mL vial, Omnirad 2100 (28.7 mg, 9 wt of total formulation weight) and lauryl mercaptan (218.6 mg, fₜₕᵢₒₗ=1, 1.08 mmoles, 72 equiv.) were weighed out to maintain an alkene:thiol ratio of 1:1. The solution of poly(3) in dichloromethane was then added to the vial containing lauryl mercaptan and further mixed by vortexing at room temperature. The formulation was irradiated with UV light (OmniCure S1500 Curing System, 100 mW/cm²) for 10 min to synthesize poly(5).

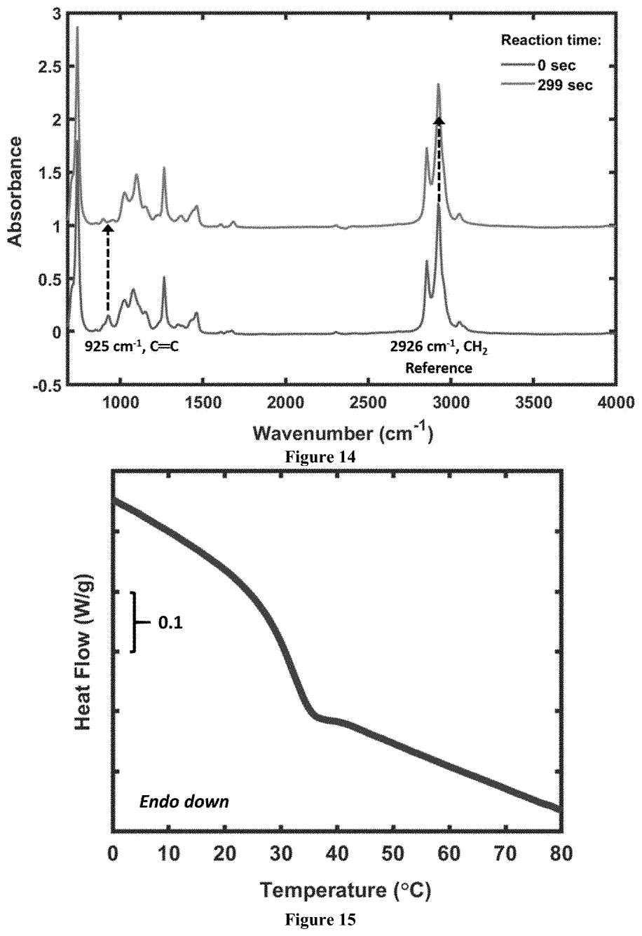

The synthesized polymer, poly(5), was isolated from the crude mixture by dialysis in dichloromethane (6 to 8 kDa RC dialysis tubing) to remove unreacted poly(3). The purified polymer was concentrated by rotary evaporation and finally the polymer was dried under vacuum overnight. The poly(5) was analyzed by RT-FTIR and the FTIR spectra is shown in FIG. 14.

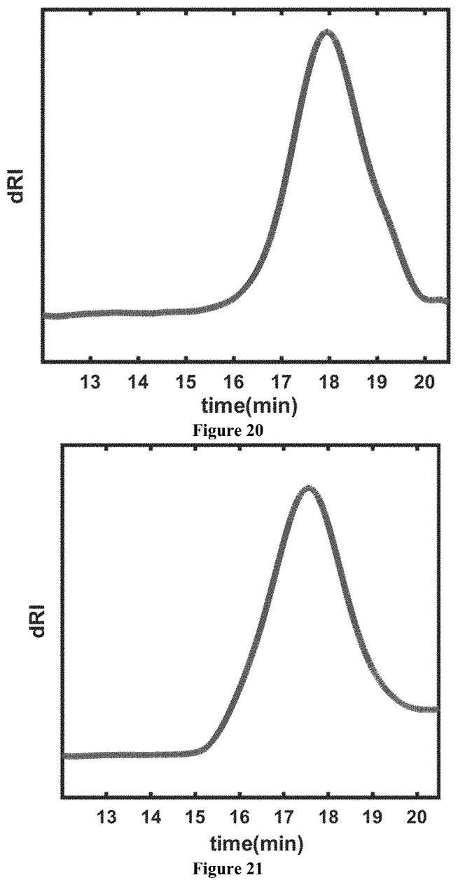

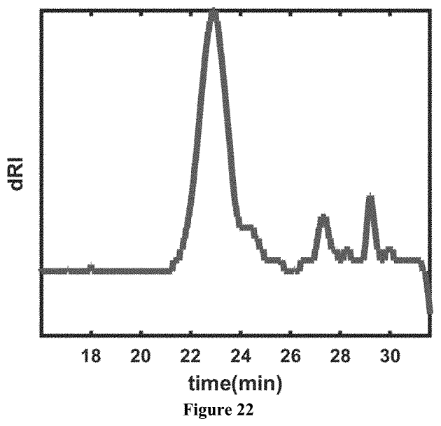

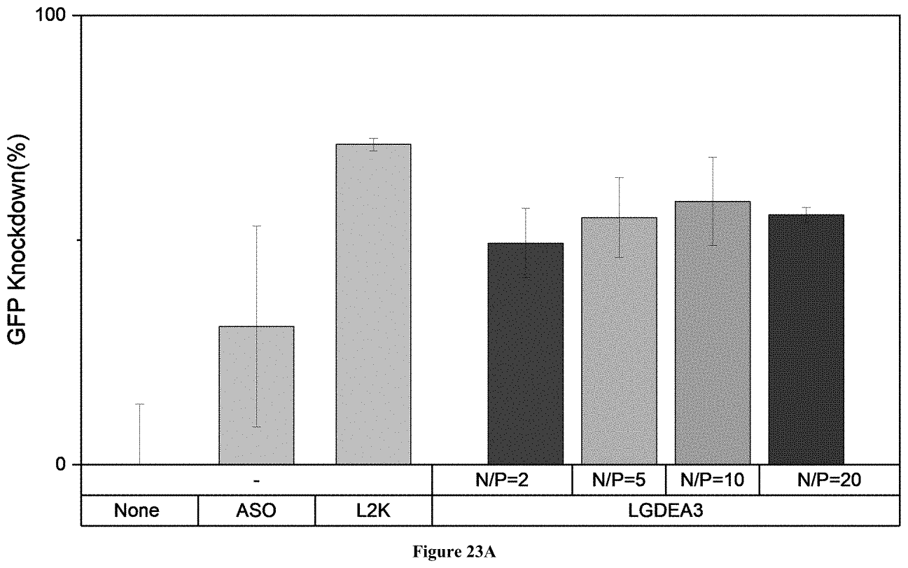

SEC analysis of poly(3), poly(4) and poly(5) was accomplished and is shown in Table 9 below. SEC traces of poly(2), poly(3), poly(4) and poly(5) are shown in FIGS. 19-22.

TABLE 9

| Polymer | poly(3) | poly(4) | poly(5) |
|---|---|---|---|
| Mₙ | 6.76 kDa | 27 kDa | 25.4 kDa |
| M_{w} | 10.5 kDa | 31.4 kDa | 37.3 kDa |
| Ð | 1.54 | 1.2 | 1.47 |

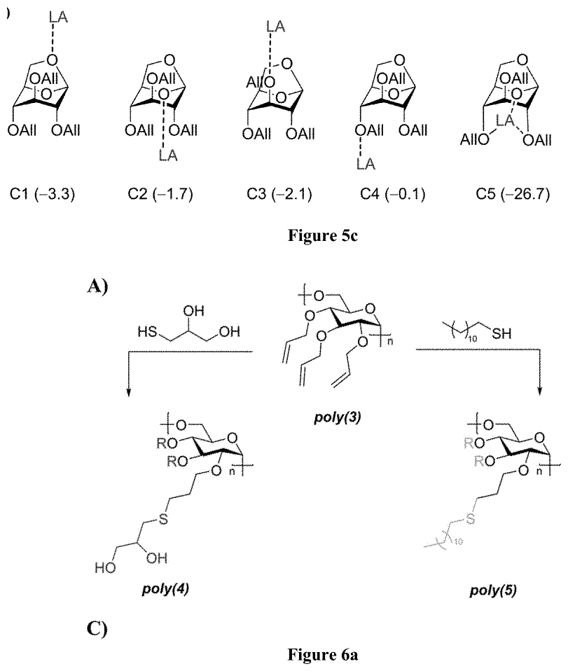

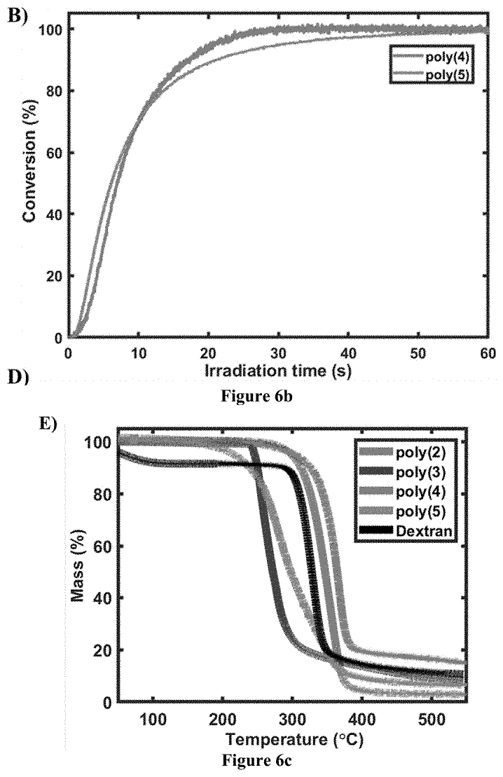

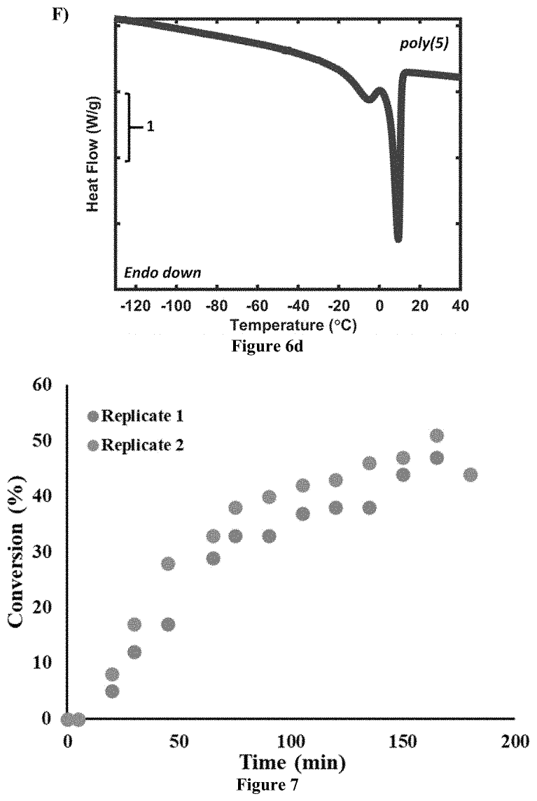

The allylic pendant groups enable facile post-polymerization modification of poly(3), which was envisioned to serve as a stereo- and regio-regular scaffold for rapid, UV-initiated thiol-ene click reactions for further tailoring polymer properties. Such a renewably-derived and stereoregular scaffold is attractive for many applications such as sustainable polymers and biologically active polymers. Thioglycerol and lauryl mercaptan were chosen as model thiols as they are structurally similar to renewable glycerol and lauryl alcohol. Additionally, the contrast in hydrophilicity/hydrophobicity of these thiols was expected to provide starkly different properties after modification (FIG. 6a). Real-time Fourier-transform infrared (RT-FTIR) spectroscopy was used to study the kinetics of thiol-ene reactions with poly(3). FIG. 6b depicts the kinetic data for the formation of poly(4) and poly(5) based on the conversion of the C═C functional groups in poly(3). Both the reactions reach a plateau conversion of >99% in −1 min, suggested near full consumption of the allylic pendant groups in poly(3). ¹H NMR analysis of purified poly(4) and poly(5) indicated complete disappearance of vinylic proton signals. Analysis of poly(4) and poly(5) by size-exclusion chromatography also showed an increase in Mₙ as compared to poly(3), but some of this increase may have been due to fractionation as a result of purification steps (Table 9).

### Example 6: Solubility of Polymers

The poly(2) is soluble in solvents with intermediate polarity, whereas poly(3) is soluble in all tested solvents except water. As expected, poly(4) and poly(5) display starkly different solubility properties owing to the contrast in hydrophilicity. The poly(4) is the only water-soluble polymer in the synthesized library, whereas poly(5) is insoluble in highly polar alcohols and DMF. Notably, most of the levoglucosan based polysaccharides are soluble in less hazardous solvents such as methyl tert-butyl ether, and green solvents such as acetone and ethyl acetate. The extensive list of solubility is shown in Table 10, wherein “x” indicates the polymer is not soluble in the solvent and “/” indicates the polymer is soluble.

The solubility of synthesized polymers was tested in a range of solvents (Table 10). As expected, poly(4) and poly(5) display starkly different solubility properties, with poly(4) being the only water-soluble polymer in the synthesized library.

TABLE 10

| Polymer/ | | | | | ε at |
|---|---|---|---|---|---|
| Solvent | poly(2) | poly(3) | poly(4) | poly(5) | 20° C. |
| Hexanes | [symbol] | ✓ | [symbol] | ✓ | 1.9 |
| MTBE | ✓ | ✓ | [symbol] | ✓ | 4.5 |
| CHCl₃ | ✓ | ✓ | [symbol] | ✓ | 4.8 |
| EtOAc | ✓ | ✓ | [symbol] | ✓ | 6.02 |
| THF | ✓ | ✓ | [symbol] | ✓ | 7.6 |
| CH₂Cl₂ | ✓ | ✓ | [symbol] | ✓ | 9.1 |
| Acetone | ✓ | ✓ | [symbol] | ✓ | 20.6 |
| Ethanol | [symbol] | ✓ | ✓ | [symbol] | 22.4 |
| Methanol | [symbol] | ✓ | ✓ | [symbol] | 32.6 |
| DMF | ✓ | ✓ | ✓ | [symbol] | 36.7 |
| Water | [symbol] | [symbol] | ✓ | [symbol] | 79.7 |

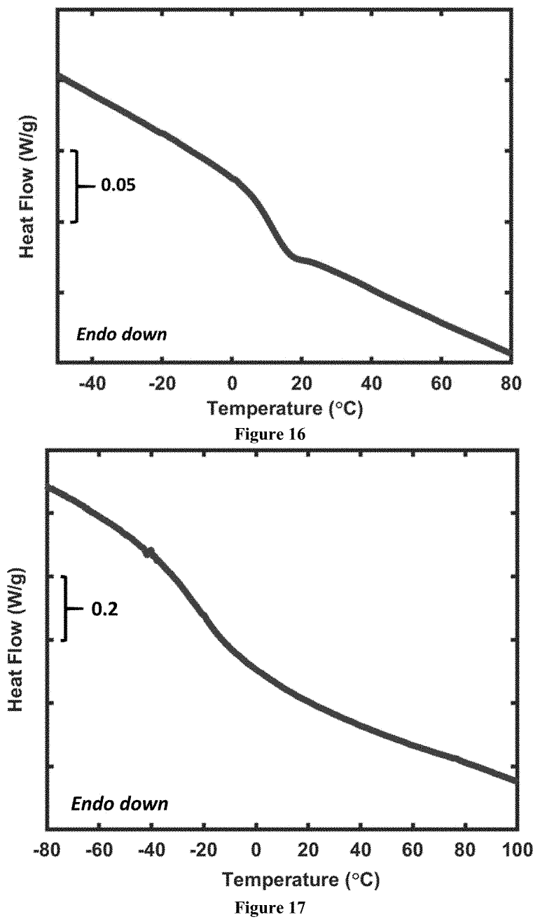

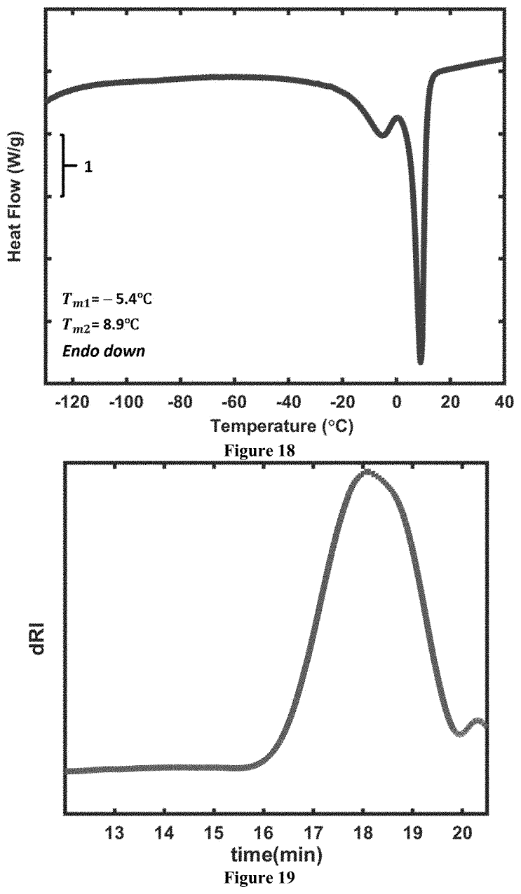

The DSC thermograms of poly(2), poly(3), poly(4), and poly(5) were taken and are shown in FIGS. 15-18.

Lastly, the thermal properties of the synthesized polysaccharides were examined via thermogravimetric analysis (TGA) and differential scanning calorimetry (DSC) to understand thermal stability and thermal transitions respectively. Dextran, a natural and commercial polysaccharide, was used as a control because it also contains 1,6-α-glycosidic linkages and glucopyranose rings. Except for poly(3), each of the homopolymers demonstrated excellent thermal stability with a T_{d,10 %}>300° C. (FIG. 6c), consistent with the thermal stability of dextran and trimethylated levoglucosan polymer (T_{d,10 %}=347° C.) (Yoshida et al., Elucidation of High Ring-Opening Polymerizability of Methylated 1,6-Anhydro Glucose, J. Polym. Sci. Part A Polym. Chem., 2009, 47, 1013-1022). Comparatively, poly(3) had a slightly lower T_{d,10 %} (250° C.), which could be attributed to the fragmentation of pendant allylic ether groups at this temperature. In general, levoglucosan-based polysaccharides possessed excellent thermal stability even at moderate molecular weights, likely due to the rigid glucopyranose ring in the backbone. The glass transition temperature (T_{g}) of poly(2), poly(3), and poly(4) was observed to be 32° C., 5° C., and −14° C., respectively (FIGS. 15-18); no T_{g} was observed for poly(5) down to −150° C., which is consistent with other lauryl-pendant polymers (Sajjad et al., Block Copolymer Pressure-Sensitive Adhesives Derived from Fatty Acids and Triacetic Acid Lactone, ACS Appl. Polym. Mater., 2020, 2, 2719-2728). The T_{g} values of poly(2), poly(3), and poly(5) were lower than that of the control dextran (199° C.), potentially due to the strong hydrogen bonding between unsubstituted dextran chains. Interestingly, poly(4) exhibited a sub-zero T_{g} despite the presence of pendant hydroxyl groups, potentially due to the added flexibility of the aliphatic methylene units. Additionally, the T_{g} for poly(2) was lower than that of trimethylated levoglucosan polymer (T_{g}˜300° C.). Overall, DSC analysis demonstrates that the T_{g} of levoglucosan-based polysaccharides can be tailored based on the identity of the pendant groups, with a remarkable T_{g} window of >180° C. accessible with the few pendant groups evaluated herein.

DSC analysis also revealed an interesting double melting peak for poly(5) as shown in FIG. 6d (Tₘ₁=−5° C., Tₘ₂=9° C.). The crystallinity of poly(5) was likely due to lauryl side chain crystallization, also observed in other polymeric systems with lauryl side chains (Sajjad et al., Block Copolymer Pressure-Sensitive Adhesives Derived from Fatty Acids and Triacetic Acid Lactone, ACS Appl. Polym. Mater., 2020, 2, 2719-2728). In addition, even at moderate molecular weights, poly(3) offered very high functionality for modification (for Mₙ=6.7 kDa the number of allyl groups=72), enabling the facile synthesis of highly functional materials. The high thermal stability, wide range of accessible T_{g} values, and the potential crystallization phenomena of these derivatives demonstrated that poly(3) is an excellent renewable scaffold for post-polymerization reactions to tailor properties to desired applications in a variety of applications. Furthermore, the levoglucosan platform provides easy access to fully functionalized dextran derivatives with the ability to install the desired pendant group both pre- and post-polymerization.

### Example 7: Preparation of Cationic Polymer and ASO Delivery

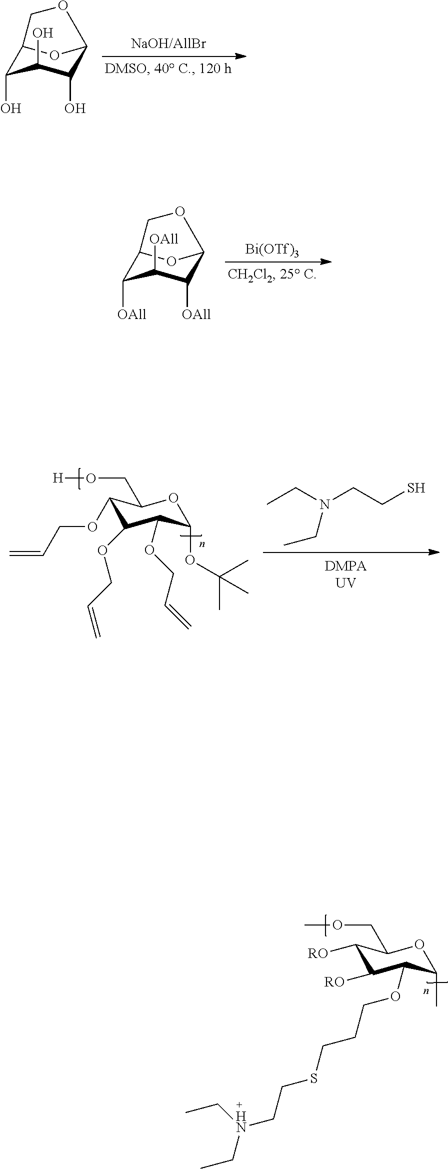

The poly(3) was prepared according to Synthetic route 2 of Example 2, and the cationic polymer, (P(DEA)₃LG), was prepared according to Examples 4 and/or 5 above. The term “DMPA” above stands for 2,2-dimethoxy-2-pheniylacetophenone. The Mₙ of poly(3) was 4200 Da. The Mₙ of the P(DEA)₃LG was 12150 Da, and D of P(DEA)₃LG was 1.3.

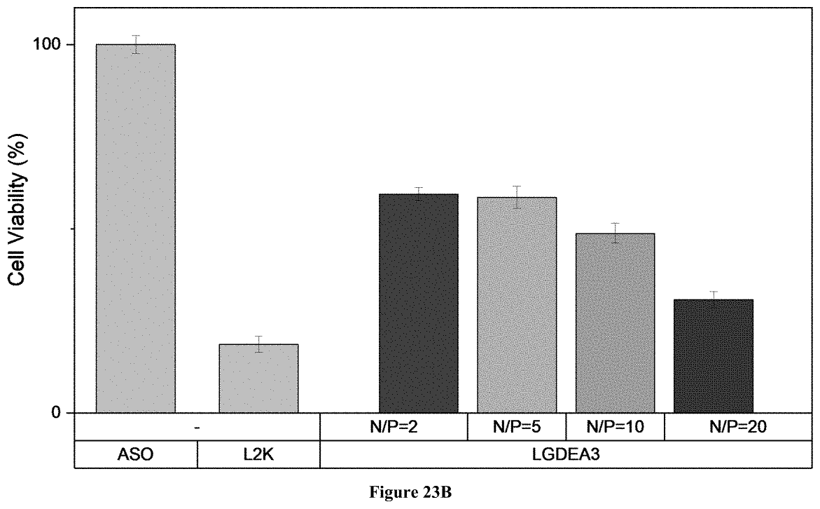

The ASO delivery experiment was accomplished similarly to the methods described in Hanson et al., Bioconjugate Chem. 2022, 33, 11, 2121-2131. Cells were plated at 50 K per well in 24 well plates with 1 mL media (1 mL Dulbeccos's Modified Eagle Medium (DMEM) with 10% fetal bovine serum (FBS) and 1% penicillin streptomycin). The plated cells were let sit overnight. A 165 μL ASO solution and 165 μL micelle solution were mixed together and let sit for 15 minutes. 660 μL of Opti-MEM™ was added to the mixture and 300 μL was added to each plated well. The wells were added to a 37° C. incubator overnight. The media was replaced 24 hours after transfection with aspirate and 1 mL Dulbeccos's Modified Eagle Medium (DMEM) with 10% fetal bovine serum (FBS) and 1% penicillin streptomycin. Lipofectamine was used as a positive control. ASOs were also used as a control. The cell viability was normalized to the ASO control. The green fluorescent protein (GFP)knockdown percentage was normalized to zero (i.e., “none” in FIG. 23A). As can be seen in FIGS. 23A and 23B, the P(DEA)₃LG ASO delivery efficiency was comparable to commercial control lipofectamine (L2K). The P(DEA)₃LG outperformed lipofectamine in cell viability.

Several embodiments have been described. Nevertheless, it will be understood that various modifications may be made without departing from the spirit and scope of disclosure. Accordingly, other embodiments are within the scope of the following claims.

What is claimed is:

1. A compound according to Formula (I): 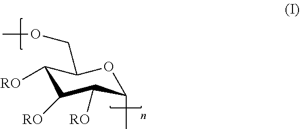
wherein:
each R is independently C₂-C₂₀ alkynyl, C₁-C₂₀ heteroalkyl, C₁-C₈ heterocycloalkyl, C₃-C₈ cycloalkyl, C₆-C₂₀ aryl, C₁-C₁₅ heteroaryl and each of the heteroalkyl, heterocycloalkyl, and heteroaryl comprise 1-5 heteroatoms selected from N, O, and S; and
n is in a range of 3 to 1000;
with the proviso that each R cannot be benzyl.

2. The compound of claim 1, wherein at least one R is independently C₂-C₂₀ alkynyl, C₁-C₂₀ heteroalkyl, C₁-C₈ heterocycloalkyl, or C₁-C₁₅ heteroaryl.

3. The compound of claim 1, wherein each R is independently C₂-C₂₀ alkynyl, C₁-C₂₀ heteroalkyl, C₁-C₈ heterocycloalkyl, or C₁-C₁₅ heteroaryl.

4. The compound of claim 1, wherein at least one R is C₁-C₂₀ heteroalkyl.

5. The compound of claim 1, wherein each R is independently C₁-C₂₀ heteroalkyl.

6. The compound of claim 1, wherein at least one R is

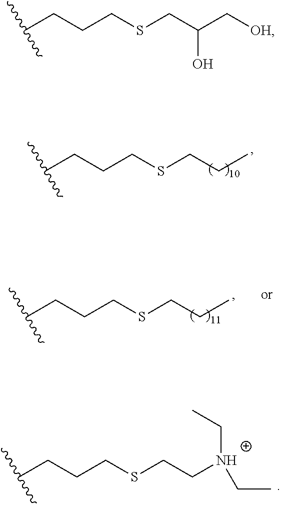

7. The compound of claim 1, wherein each R is

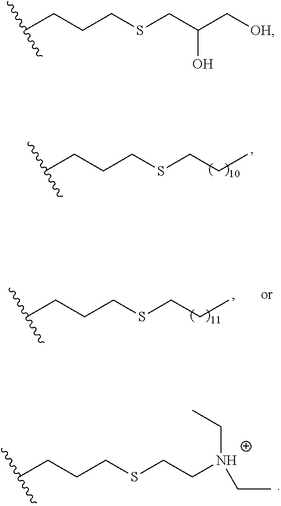

8. The compound of claim 1, wherein at least one R is

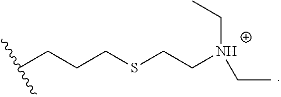 [SMILES: *CCCSCC[NH+](CC)CC]

9. The compound of claim 1, wherein each R is

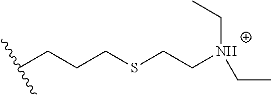 [SMILES: *CCCSCC[NH+](CC)CC]

10. A method of modifying a polymer, the method comprising:
combining a thiol or an azide, and a compound according to Formula (I): 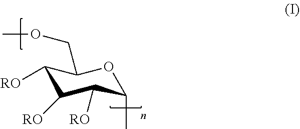
to form a compound according to Formula (II): 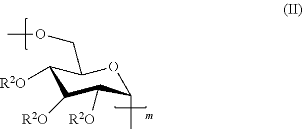
wherein:
each R is independently C₂-C₂₀ alkenyl, C₂-C₂₀ alkynyl, C₁-C₂₀ heteroalkyl, C₁-C₈ heterocycloalkyl, C₁-C₁₅ heteroaryl and each of the heteroalkyl, heterocycloalkyl, and heteroaryl comprise 1-5 heteroatoms selected from N, O, and S;
each R² is independently C₂-C₂₀ alkyl, C₂-C₂₀ alkylene, C₂-C₂₀ alkylyne, C₁-C₂₀ heteroalkyl, C₁-C₈ heterocycloalkyl, C₂-C₂₀ heteroalkylene, C₂-C₂₀ heteroalkylyne, C₃-C₈ cycloalkyl, C₆-C₂₀ aryl, C₁-C₁₅ heteroaryl and each of the heteroalkyl, heterocycloalkyl, heteroalkylene, heteroalkylyne, and heteroaryl comprise 1-5 heteroatoms selected from N, O, and S;
at least one R² is a C₁-C₂₀ thioalkyl or C₁-C₁₅ heteroaryl; and
n and m are each independently in a range of 3 to 1000.

11. The method of claim 10, wherein at least one R is

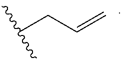 [SMILES: *CC=C]

12. The method of claim 10, wherein at least one R² is a C₁-C₂₀ thioalkyl.

13. The method of claim 10, wherein at least one R² is

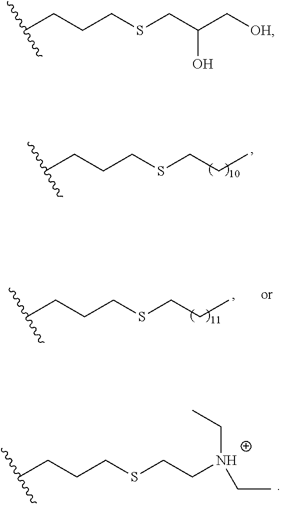

14. The method of claim 10, wherein at least one R² is C₁-C₁₅ heteroaryl.

15. The method of claim 14, wherein the R² is triazolyl.

16. The compound of claim 1, wherein at least one R is C₁-C₂₀ heteroalkyl and wherein each C₁-C₂₀ heteroalkyl comprises 1-3 heteroatoms selected from S and N.

17. The compound of claim 1, wherein the compound of Formula (I) is cationic.

18. The method of claim 10, wherein at least one R² is C₁-C₂₀ heteroalkyl and wherein each C₁-C₂₀ heteroalkyl comprises 1-3 heteroatoms selected from S and N.

19. A pharmaceutical composition comprising a compound of claim 1 or a pharmaceutically acceptable salt thereof and an antisense oligonucleotide.

20. A method of delivering an antisense oligonucleotide to a subject comprising administering to the subject a therapeutically effective amount of the pharmaceutical composition of claim 19.
